(19) United States
(12) Patent Application Publication
(10) Pub. No.: US 2025/0003971 A1
(43) Pub. Date: Jan. 2, 2025

(54) METHOD FOR PROVIDING INFORMATION PERTAINING TO CANCER, SYSTEM FOR PROVIDING INFORMATION PERTAINING TO CANCER, AND METHOD FOR TREATING CANCER

(71) Applicants: KAGAMI INC., Ibaraki-shi, Osaka (JP); KEIO UNIVERSITY, Minato-ku, Tokyo (JP)

(72) Inventors: Tomohisa SUJINO, Tokyo (JP); Takanori KANAI, Tokyo (JP); Kai TSUGARU, Tokyo (JP); Jumpei SASABE, Tokyo (JP); Masashi MITA, Ibaraki-shi, Osaka (JP)

(73) Assignees: KAGAMI INC., Ibaraki-shi, Osaka (JP); KEIO UNIVERSITY, Minato-ku, Tokyo (JP)

(21) Appl. No.: 18/688,730

(22) PCT Filed: Sep. 5, 2022
(86) PCT No.: PCT/JP2022/033332

§ 371 Date: Mar. 1, 2024

(30) Foreign Application Priority Data
Sep. 3, 2021 (JP) 2021-144350

(51) Int. Cl. G01N 33/574

(52) U.S. Cl. CPC G01N 33/57488

(57) ABSTRACT

There are many options in cancer evaluation and treatment, and it is desired to ascertain the characteristics and conditions of individual patients and improve treatment efficacy and cost performance. Provided is a method for providing information pertaining to a cancer in a subject using an indicator based on the amount of D-amino acid in a biological sample from the subject, wherein the information is selected from the group consisting of: a verification result of the validity of a test or diagnosis result of the cancer in the subject; a classification of the degree of progression of the cancer in the subject; a result of a prognosis prediction for the cancer in the subject; and information for selecting a treatment means for the cancer in the subject. Also provided is a system for providing information pertaining to a cancer in a subject. Further provided is a cancer treatment method for treating a subject via a cancer treatment means selected on the basis of the information provided by the method or the system.

### FIELD

The present invention relates to a method for providing information associated with cancer and to a system for providing information associated with cancer. The invention further relates to a method for treating cancer.

### BACKGROUND

Cells that proliferate autonomously without normal regulatory control due to a genetic abnormality in the cells are collectively referred to as a tumor, and when they have undergone invasive cell proliferation and metastasis they are referred to as cancer (malignant tumor). Cancer treatment methods include surgery, radiotherapy, chemotherapy, drug therapy and immunotherapy, which are carried out either alone or in appropriately selected combinations as multidisciplinary treatment. As a result of progress in cancer research since the late 1990s it has become clear that drug response in chemotherapy and drug therapy, including both effects and side-effects, differs depending on the nature of the cancer, and therefore markers (biomarkers) have been developed for selection of optimal drugs. Biomarkers include those used to confirm individual features or types for determining changes in proteins or genes in blood, urine, saliva, cells and tissues, and tumor markers used in health examination or complete medical checkups. Current biomarkers being used include EGFR gene mutations and ALK fused gene in lung cancer, as well as HER2 protein overexpression in breast cancer or stomach cancer, in connection with the action mechanisms of drugs which target molecules associated with tumor proliferation (molecular targeted drugs). For example, EGFR gene mutation markers are used to predict the therapeutic effect of EGFR tyrosine kinase inhibitors (EGFR inhibitors). Immune checkpoint inhibitors are also being researched as candidate markers, including microsatellite instability, tumor mutation burden, PD-L1 positivity rate and Epstein-Barr virus.

Recent advances in quantitative research on distinguishing trace D-amino acids and L-amino acids in living bodies including mammals, owing to higher level performance of techniques used to identify and analyze chiral amino acids, has led to elucidation of the presence and function of some D-amino acids which were previously treated as total amino acids (D-amino acids+L-amino acids), or as L-amino acids for convenience, due to technical limitations of the prior art. It has been reported that D-amino acids are present in varying levels in living bodies, tissues, cells and body fluids depending on effects such as intake, symbiotic bacteria, metabolism (decomposition and synthesis), transport and excretion (NPLs 1 to 5), that a characteristic chiral amino acid profile is exhibited in diseases such as kidney disease and other physical conditions (PTL 1), that D-amino acids are involved in intestinal immunity (NPL 6) and protect kidney-derived cells (NPL 2), and that carbohydrate metabolism in neurons is involved in D-serine biosynthesis (NPL 7). It has been disclosed that cancer patient blood shows fluctuations in D-serine, D-threonine, D-alanine, D-asparagine, D-allothreonine, D-glutamine, D-proline and D-phenylalanine in kidney cancer, D-histidine and D-asparagine in prostate cancer and D-alanine in lung cancer (PTL 1). However, evaluation of individual cancer patients in a stratified manner based on D-amino acid levels has not yet been implemented.

### CITATION LIST

### Patent Literature

[PTL 1] International Patent Publication No. WO2013/140785
[PTL 2] Japanese Unexamined Patent Publication No. 2008-96313
[PTL 3] International Patent Publication No. WO2020/196436

### Non Patent Literature

[NPL 1] Y. Miyoshi, R. Konno, J. Sasabe, K. Ueno, Y. Tojo, M. Mita, S. Aiso and K. Hamase, Alteration of intrinsic amounts of D-serine in the mice lacking serine racemase and D-amino acid oxidase, Amino Acids, 43,1919-1931 (2012). DOI: 10.1007/s00726-012-1398-4
[NPL 2] Y. Nakade, Y. Iwata, K. Furuichi, M. Mita, K. Hamase, R. Konno, T. Miyake, N. Sakai, S. Kitajima, T. Toyama, Y. Shinozaki, A. Sagara, T. Miyagawa, A. Hara, M. Shimizu, Y. Kamikawa, K. Sato, M. Oshima, S. Yoneda-Nakagawa, Y. Yamamura, S. Kaneko, T. Miyamoto, M. Katane, H. Homma, H. Morita, W. Suda, M. Hattori and T. Wada, Gut microbiota-derived D-serine protects against acute kidney injury, JCI Insight, 3 (20) (2018). DOI: 10.1172/jci.insight.97957
[NPL 3] M. Ariyoshi, M. Katane, K. Hamase, Y. Miyoshi, M. Nakane, A. Hoshino, Y. Okawa, Y. Mita, S. Kaimoto, M. Uchihashi, K. Fukai, K. Ono, S. Tateishi, D. Hato, R. Yamanaka, S. Honda, Y. Fushimura, E. Iwai-Kanai, N. Ishihara, M. Mita, H. Homma and S. Matoba, D-Glutamate is metabolized in the heart mitochondria, Scientific Reports, 7,43911 (2017). DOI: 10.1038/srep43911
[NPL 4] P. Wiriyasermkul, S. Moriyama, Y. Tanaka, P. Kongpracha, N. Nakamae, M. Suzuki, T. Kimura, M. Mita, J. Sasabe and S. Nagamori, D-Serine, an emerging biomarker of kidney diseases, is a hidden substrate of sodium-coupled monocarboxylate transporters, bioRxiv preprint. DOI: 10.1101/2020.08.10.244822
[NPL 5] A. Hesaka, S. Sakai, K. Hamase, T. Ikeda, R. Matsui, M. Mita, M. Horio, Y. Isaka and T. Kimura, D-Serine reflects kidney function and diseases, Scientific Reports, 9,5104 (2019). DOI: 10.1038/s41598-019-41608-0
[NPL 6] J. Sasabe, Y. Miyoshi, S. Rakoff-Nahoum, T. Zhang, M. Mita, B. M. Davis, K. Hamase and M. K. Waldor, Interplay between microbial D-amino acids and host D-amino acid oxidase modifies murine mucosal defence and gut microbiota, Nature Microbiology, 1,16125 (2016). DOI: 10.1038/nmicrobiol.2016.125
[NPL 7] M. Suzuki, J. Sasabe, Y. Miyoshi, K. Kuwasako, Y. Muto, K. Hamase, M. Matsuoka, N. Imanishi and S. Aiso, Glycolytic flux controls D-serine synthesis through glyceraldehyde-3-phosphate dehydrogenase in astrocytes, Proceedings of the National Academy of Sciences of the United States of America, 112 (17), E2217-E2224 (2015). DOI: 10.1073/pnas.1416117112
[NPL 8] N. Okamoto, Y. Miyagi, A. Chiba, M. Akaike, M. Shiozawa, A. Imaizumi, H. Yamamoto, T. Ando, M. Yamakado and O. Tochikubo, Diagnostic modeling with differences in plasma amino acid profiles between non-cachectic colorectal/breast cancer patients and healthy individuals, International Journal of Medicine and Medical Sciences, 1 (1), 001-008 (2009). DOI: 10.5897/IJMMS.90,00074
[NPL 9] Okamoto, N., “Cancer screening using AminoIndex technology”, Ningen Dock, 26 (3), 454-466 (2011).DOI:10.11320/ningendock.26.454

### SUMMARY

### Technical Problem

Cancer is the number one cause of death, and while many treatment alternatives exist including surgery, radiotherapy, chemotherapy, drug therapy and immunotherapy, it is still desirable to develop improved methods with greater therapeutic effects and better cost efficiency based on the features and conditions ascertained for individual patients.

### Solution to Problem

The present inventors have comprehensively quantified and analyzed chiral amino acids (D-amino acids and L-amino acids) in cancer patient blood, and have found chiral amino acid levels in blood to be correlated with pathology, disease stage and prognosis, and therapeutic effect. As a result of much diligent research in this regard, it was found that developing indicators based on D-amino acids is clinically useful for diagnosis and disease staging, prognosis prediction, and treatment method selection, and the present invention was developed to provide a feasible solution method. Specifically, the present invention encompasses the following.

[1] A method for providing information associated with cancer in a subject, using an indicator based on the amount of a D-amino acid in a biological sample (such as blood, urine or feces) from the subject, wherein the information is selected from the group consisting of:
a validation result for a cancer examination result or a cancer diagnosis result for the subject,
classification of cancer progression for the subject,
a result of cancer prognosis prediction for the subject, and
information for selection of cancer treatment means for the subject.

[2] The method according to case [1] above, wherein the indicator is a formula or value obtained by correcting the amount of the D-amino acid with an in vivo substance in the subject.

[3] The method according to case [2] above, wherein the in vivo substance is an L-amino acid.

[4] The method according to case [1] above, wherein the indicator is a formula or value obtained by correcting the amount of the D-amino acid with a kidney function marker of the subject.

[5] The method according to case [4] above, wherein the kidney function marker is the amount of one or more factors selected from the group consisting of creatinine, cystatin C, inulin clearance, creatinine clearance, urine protein, urine albumin, β2-MG, α1-MG, NAG, L-FABP and NGAL.

[6] The method according to any one of cases [1] to [5] above, wherein the D-amino acid is one or more selected from the group consisting of D-proline, D-serine, D-alanine, D-asparagine and D-leucine.

[7] The method according to any one of cases [1] to [6] above, wherein the cancer is gastrointestinal cancer.

[8] The method according to case [7] above, wherein the gastrointestinal cancer is stomach cancer, esophageal cancer or colorectal cancer.

[9] The method according to any one of cases [1] to [8] above, wherein the cancer treatment means is an antineoplastic agent.

[10] The method according to case [9] above, wherein the antineoplastic agent is an immune checkpoint inhibitor and/or an NMDA receptor antagonist.

[11] The method according to case [10] above, wherein the immune checkpoint inhibitor is an inhibitor of an immune checkpoint molecule selected from the group consisting of CTLA-4, PD-1, PD-L1, PD-L2, LAG-3, TIM3, BTLA, B7H3, B7H4, 2B4, CD160, A2aR, KIR, VISTA and TIGIT.

[12] The method according to any one of cases [1] to [11] above, wherein the validation result for the cancer examination result or the diagnosis result for the subject are provided by comparing the indicator with a determination value determined from the amounts of D-amino acids in biological samples from subjects having cancer.

[13] The method according to any one of cases [1] to [11] above, wherein the information for selection of cancer treatment means for the subject is provided by comparing the indicator with a determination value determined from the amounts of D-amino acids in biological samples from patients with cancer that respond and/or do not respond to cancer treatment means.

[14] The method according to any one of cases [1] to [11] above, wherein the information relating to classification of cancer progression for the subject is provided by comparing the indicator with an determination value determined from the amounts of D-amino acids in biological samples from patients with cancer whose cancer stage has been classified.

[15] The method according to any one of cases [1] to [11] above, wherein the information relating to prognosis prediction of cancer for the subject is provided by comparing the indicator with an determination value determined from the amounts of D-amino acids in biological samples from patients with cancer with information regarding prognosis.

[16] A method for treating cancer, wherein a subject is treated by cancer treatment means selected based on information provided by the method according to any one of cases [1] to [15] above.

[17] The method according to case [16] above, wherein the cancer is gastrointestinal cancer.

[18] The method according to case [17] above, wherein the gastrointestinal cancer is stomach cancer, esophageal cancer or colorectal cancer.

[19] The method according to any one of cases [16] to [18] above, wherein the cancer treatment means is an antineoplastic agent.

[20] The method according to case [19] above, wherein the antineoplastic agent is an immune checkpoint inhibitor.

[21] The method according to case [20] above, wherein the immune checkpoint inhibitor is an inhibitor of an immune checkpoint molecule selected from the group consisting of CTLA-4, PD-1, PD-L1, PD-L2, LAG-3, TIM3, BTLA, B7H3, B7H4, 2B4, CD160, A2aR, KIR, VISTA and TIGIT.

[22] The method according to any one of cases [16] to [21] above, wherein the cancer treatment means consists of or includes means for adjusting the amount of the D-amino acid in a biological sample of a subject, so that the value of an indicator based on the amount of the D-amino acid in the biological sample of the subject is within or near a prescribed range.

[23] The method according to case [22] above, wherein the means for adjusting the amount of the D-amino acid is administration of the D-amino acid or its pharmaceutically acceptable salt, or administration of a composition from which the D-amino acid or its pharmaceutically acceptable salt has been removed.

[24] The method according to any one of cases [16] to [23] above, wherein the cancer treatment means is an NMDA receptor antagonist.

[25] The method according to case [24] above, wherein the NNMDA receptor antagonist is memantine or its pharmaceutically acceptable salt.

[26] A system for providing information associated with cancer in a subject, comprising a memory unit, an input unit, an analytical measurement unit, a data processing unit and an output unit, wherein:
the memory unit stores the amount of a D-amino acid in a biological sample (such as blood, urine or feces) and a determination value associated with cancer, the determination value being selected from the group consisting of:
an determination value relating to the validity of cancer examination results or cancer diagnosis results determined from the amounts of D-amino acids in the biological samples of patients with cancer;
an determination value relating to cancer progression determined from the amounts of the D-amino acids in the biological samples of the patients with cancer;
an determination value relating to cancer prognosis determined from the amount of the D-amino acid in the biological sample of the patient with cancer; and
an determination value for selection of cancer treatment means determined from the amount of the D-amino acids in the biological samples of the patients with cancer;
the analytical measurement unit separates and quantifies the D-amino acid in the biological sample of the subject;
the data processing unit selects information associated with cancer in the subject by comparing an indicator based on the amount of the D-amino acid of the subject with the determination value stored in the memory unit; and
the output unit outputs the information.

[27] The system according to case [26] above, wherein the indicator is a formula or value obtained by correcting the amount of the D-amino acid with an in vivo substance in the subject.

[28] The system according to case [27] above, wherein the in vivo substance is an L-amino acid.

[29] The system according to case [26] above, wherein the indicator is a formula or value obtained by correcting the amount of the D-amino acid with a kidney function marker of the subject.

[30] The system according to case [29] above, wherein the kidney function marker is the amount of one or more factors selected from the group consisting of creatinine, cystatin C, inulin clearance, creatinine clearance, urine protein, urine albumin, β2-MG, α1-MG, NAG, L-FABP and NGAL.

[31] The system according to any one of cases [26] to [30] above, wherein the D-amino acid is one or more selected from the group consisting of D-proline, D-serine, D-alanine, D-asparagine and D-leucine.

[32] The system according to any one of cases [26] to [31] above, wherein the cancer is gastrointestinal cancer.

[33] The system according to case [32] above, wherein the gastrointestinal cancer is stomach cancer, esophageal cancer or colorectal cancer.

[34] The system according to any one of cases [26] to [33] above, wherein the cancer treatment means is an antineoplastic agent.

[35] The system according to case [34] above, wherein the antineoplastic agent is an immune checkpoint inhibitor.

[36] The system according to case [35] above, wherein the immune checkpoint inhibitor is an inhibitor of an immune checkpoint molecule selected from the group consisting of CTLA-4, PD-1, PD-L1, PD-L2, LAG-3, TIM3, BTLA, B7H3, B7H4, 2B4, CD160, A2aR, KIR, VISTA and TIGIT.

[37] The system according to any one of cases [26] to [36] above, wherein the validation result for cancer examination or diagnosis results for the subject are provided by comparing the indicator with a determination value determined from the amount of a D-amino acids in the biological samples from the patients with cancer.

[38] The system according to any one of cases [26] to [36] above, wherein the information for selection of cancer treatment means for the subject is provided by comparing the indicator with an determination value determined from the amounts of the D-amino acids in the biological samples from the patients with cancer that responds and/or does not respond to cancer treatment means.

[39] The system according to any one of cases [26] to [36] above, wherein the information relating to classification of cancer progression for the subject is provided by comparing the indicator with an determination value determined from the amounts of the D-amino acids in the biological samples from the patients with cancer with information regarding prognosis.

[40] The system according to any one of cases [26] to [36] above, wherein the information relating to prognosis prediction of cancer for the subject is provided by comparing the indicator with an determination value determined from the amounts of the D-amino acids in the biological samples from the patients with cancer with information regarding prognosis.

### Advantageous Effects of Invention

According to the invention it is possible to use an indicator based on the amount of the D-amino acid of a subject with cancer to analyze, extract, study, select or provide a suitable method of treatment on a personal level, thereby helping to control effects and side-effects, so as to achieve precision medicine for improved patient QOL, and to lower medical costs.

### BRIEF DESCRIPTION OF DRAWINGS

FIG. 1 shows the amount of D-amino acids 1 in blood plasma harvested from healthy subjects and subjects with stomach cancer or esophageal cancer.

FIG. 2 shows ROC curves for outcomes for presence or absence of cancer, with the amounts of D-serine or D-alanine in blood plasma as explanatory variables.

FIG. 3 shows an ROC curve for outcome for presence or absence of cancer, with a formula for the amounts of D-serine and D-alanine in blood plasma as the explanatory variable.

FIG. 4 shows percentages of D-amino acid (% D) in the total amount of each amino acid in plasma harvested from healthy subjects and subjects with stomach cancer or esophageal cancer.

FIG. 5 shows ROC curves for outcome for presence or absence of cancer, with % D-alanine in plasma as the explanatory variable.

FIG. 6 shows an ROC curve for outcome for presence or absence of cancer, with a formula for % D-serine and % D-asparagine in plasma as the explanatory variable.

FIG. 7 shows values for the amounts of D-amino acid in plasma harvested from healthy subjects and subjects with stomach cancer, after correction with creatinine (D-AA/Cre).

FIG. 8 shows ROC curves for outcomes for presence or absence of cancer, with plasma D-serine/Cre or D-alanine/Cre as explanatory variables.

FIG. 9 shows an ROC curve for outcome for presence or absence of cancer, with a formula for plasma D-serine/Cre and D-alanine/Cre as the explanatory variable.

FIG. 10 shows the amounts of D-amino acid in blood plasma harvested from healthy subjects and subjects with stomach cancer at different stages.

FIG. 11 shows percentages of D-amino acid (% D) in the total amount of each amino acid in plasma harvested from healthy subjects and subjects with stomach cancer at different stages.

FIG. 12 shows an ROC curve for outcome for success of nivolumab administration to subjects with stomach cancer, with the amount of D-serine in blood plasma as the explanatory variable.

FIG. 13 shows an ROC curve for outcome for success of nivolumab administration to a subject with stomach cancer, with a formula for the amounts of D-serine, D-asparagine, D-proline and L-alanine in blood plasma as the explanatory variable.

FIG. 14 shows an ROC curve for outcome of non-response to nivolumab administration in subjects with stomach cancer, with a formula for the amount of D-serine and D-alanine in blood plasma as the explanatory variable.

FIG. 15 shows survival curves for progression-free survival or overall survival, with nivolumab administration to subjects with stomach cancer, with a formula for the amount of D-serine and D-alanine in blood plasma as the explanatory variable.

FIG. 16 shows tumor volume and weight by control of in vivo D-amino acid levels in cancer-grafted mice.

FIG. 17 is a block diagram of the system of the invention.

FIG. 18 shows tumor volume by control of active amounts of in vivo D-amino acids (with and without memantine addition) in cancer-grafted mice.

FIG. 19 shows the amounts of D-amino acid in urine (corrected with creatinine (Cr)) harvested from healthy subjects and subjects with stomach cancer at different stages (top), and excretion rates (FED-Ser) (bottom).

FIG. 20 shows the amounts of D-Leu or L-Leu (corrected with creatinine (Cr)) in urine harvested from healthy subjects and subjects with stomach cancer at different stages.

FIG. 21 shows the amounts of L-amino acid (corrected with creatinine (Cr)) (Cr-corrected) in urine harvested from healthy subjects and subjects with stomach cancer at different stages (top), and excretion rates (FED-Ser) (bottom).

FIG. 22 shows % D-amino acids, the amounts of D-amino acid (nmol/g) and L-amino acid (nmol/g) in feces.

FIG. 23 shows the amounts of D-amino acid (top) and percentages of D-amino acid in the total amount of each amino acid (% D) (bottom) in plasma harvested from healthy subjects and subjects with stomach cancer at different stages.

FIG. 24 is a correlation diagram plotting the amounts of D-amino acid in blood plasma harvested from healthy subjects and subjects with stomach cancer at different stages, with eGFR values.

### DESCRIPTION OF EMBODIMENTS

Embodiments for carrying out the invention will be described in detail below, with the understanding that the technical scope of the invention is not limited only to these embodiments. The prior art documents cited throughout are incorporated herein by reference.

The present invention provides, as a novel evaluation approach for cancer, a method of improving diagnosis precision and assisting selection of appropriate treatment means using an indicator based on the amount of a D-amino acid in a biological sample.

According to one embodiment, the invention provides a method for providing information associated with cancer in a subject, using an indicator based on the amount of a D-amino acid in a biological sample (such as blood, urine or feces) from the subject, wherein the information is selected from the group consisting of:
a validation result for a cancer examination result or a cancer diagnosis result for the subject,
classification of cancer progression for the subject,
a result of cancer prognosis prediction for the subject, and
information for selection of cancer treatment means for the subject.

Throughout the present specification, the term “cancer” is not particularly restricted and includes, for example, leukemia (such as acute myelocytic leukemia, chronic myelogenous leukemia, acute lymphocytic leukemia and chronic lymphatic leukemia), malignant lymphoma (Hodgkin's lymphoma, non-Hodgkin lymphoma (such as adult T cell leukemia, follicular lymphoma and diffuse large B-cell lymphoma)), multiple myeloma, myelodysplastic syndrome, head and neck cancer, gastrointestinal cancer (such as esophageal cancer, esophageal adenocarcinoma, stomach cancer, colorectal cancer, colon cancer and rectum cancer), liver cancer (such as hepatocellular carcinoma), gallbladder/cholangiocarcinoma, biliary tract cancer, pancreatic cancer, thyroid cancer, lung cancer (such as non-small-cell lung carcinoma (including squamous epithelium non-small-cell lung carcinoma and non-squamous non-small cell lung carcinoma) and small-cell lung cancer), breast cancer, ovarian cancer (such as serous ovarian cancer), cervical cancer, uterine cancer, endometrial cancer, vaginal cancer, vulvar cancer, renal carcinoma (such as renal cell carcinoma), urothelial cancer (such as bladder cancer and upper urinary tract cancer), prostate cancer, testicular cancer (such as germ cell tumor), bone/soft tissue sarcoma, skin cancer (such as uveal melanoma, malignant melanoma and Merkel cell carcinoma), glioma, brain tumor (such as glioblastoma), pleural mesothelioma and cancer of unknown origin. Cancer to which the present invention is to be applied may be gastrointestinal cancer, for example, and preferably stomach cancer, esophageal cancer or colorectal cancer.

The term “D-amino acids” is used herein to include amino acids that are the “D-form” of protein constituent amino acids, as stereoisomers of amino acids that are constituents of “L-form” proteins, as well as glycine which has no stereoisomer, and specifically they include glycine, D-alanine, D-histidine, D-isoleucine, D-alloisoleucine, D-leucine, D-lysine, D-methionine, D-phenylalanine, D-threonine, D-allothreonine, D-tryptophan, D-valine, D-arginine, D-cysteine, D-glutamine, D-proline, D-tyrosine, D-aspartic acid, D-asparagine, D-glutamic acid and D-serine. Since D-cysteine in a biological sample is oxidized ex vivo and converted to D-cystine, one embodiment of the invention allows measurement of D-cystine instead of D-cysteine to determine the amount of D-cysteine in the biological sample.

The “amount of D-amino in a biological sample” referred to herein may be the amount of the D-amino acid in a specified amount of biological sample (such as blood, urine or feces), or it may the concentration. The amount of the D-amino acid in a biological sample is measured as the amount in a harvested biological sample that has been treated by centrifugal separation, sedimentation separation or other pretreatment for analysis. Therefore, the amount of the D-amino acid in the biological sample can be measured as the amount in a harvested biological sample (for example, a blood sample such as whole blood, serum or blood plasma; urine; or feces). For analysis using HPLC, as one example, the amount of the D-amino acid in a predetermined amount of a biological sample may be represented in a chromatogram, and the peak heights, areas and shapes may be quantified by analysis based on standard sample comparison and correction.

The amount of a D-amino acid and/or L-amino acid may be measured by any method, such as chiral column chromatography, or measurement using an enzyme method, or quantitation by an immunological method using a monoclonal antibody that distinguishes between optical isomers of amino acids. Measurement of the amount of a D-amino acid and/or L-amino acid in a sample according to the invention may be carried out using any method well known to those skilled in the art. Examples include chromatographic and enzyme methods (Y. Nagata et al., Clinical Science, 73 (1987), 105. Analytical Biochemistry, 150 (1985), 238., A. D'Aniello et al., Comparative Biochemistry and Physiology Part B, 66 (1980), 319. Journal of Neurochemistry, 29 (1977), 1053., A. Berneman et al., Journal of Microbial & Biochemical Technology, 2 (2010), 139., W. G. Gutheil et al., Analytical Biochemistry, 287 (2000), 196., G. Molla et al., Methods in Molecular Biology, 794 (2012), 273., T. Ito et al., Analytical Biochemistry, 371 (2007), 167.), antibody methods (T. Ohgusu et al., Analytical Biochemistry, 357 (2006), 15), gas chromatography (GC) (H. Hasegawa et al., Journal of Mass Spectrometry, 46 (2011), 502., M. C. Waldhier et al., Analytical and Bioanalytical Chemistry, 394 (2009), 695., A. Hashimoto, T. Nishikawa et al., FEBS Letters, 296 (1992), 33., H. Bruckner and A. Schieber, Biomedical Chromatography, 15 (2001), 166., M. Junge et al., Chirality, 19 (2007), 228., M. C. Waldhier et al., Journal of Chromatography A, 1218 (2011), 4537), capillary electrophoresis methods (CE) (H. Miao et al., Analytical Chemistry, 77 (2005), 7190., D. L. Kirschner et al., Analytical Chemistry, 79 (2007), 736., F. Kitagawa, K. Otsuka, Journal of Chromatography B, 879 (2011), 3078., G. Thorsen and J. Bergquist, Journal of Chromatography B, 745 (2000), 389), and high-performance liquid chromatography (HPLC) HPLC) (N. Nimura and T. Kinoshita, Journal of Chromatography, 352 (1986), 169., A. Hashimoto et al., Journal of Chromatography, 582 (1992), 41., H. Bruckner et al., Journal of Chromatography A, 666 (1994), 259., N. Nimura et al., Analytical Biochemistry, 315 (2003), 262., C. Muller et al., Journal of Chromatography A, 1324 (2014), 109., S. Einarsson et al., Analytical Chemistry, 59 (1987), 1191., E. Okuma and H. Abe, Journal of Chromatography B, 660 (1994), 243., Y. Gogami et al., Journal of Chromatography B, 879 (2011), 3259., Y. Nagata et al., Journal of Chromatography, 575 (1992), 147., S. A. Fuchs et al., Clinical Chemistry, 54 (2008), 1443., D. Gordes et al., Amino Acids, 40 (2011), 553., D. Jin et al., Analytical Biochemistry, 269 (1999), 124., J. Z. Min et al., Journal of Chromatography B, 879 (2011), 3220., T. Sakamoto et al., Analytical and Bioanalytical Chemistry, 408 (2016), 517., W. F. Visser et al., Journal of Chromatography A, 1218 (2011), 7130., Y. Xing et al., Analytical and Bioanalytical Chemistry, 408 (2016), 141., K. Imai et al., Biomedical Chromatography, 9 (1995), 106., T. Fukushima et al., Biomedical Chromatography, 9 (1995), 10., R. J. Reischl et al., Journal of Chromatography A, 1218 (2011), 8379., R. J. Reischl and W. Lindner, Journal of Chromatography A, 1269 (2012), 262., S. Karakawa et al., Journal of Pharmaceutical and Biomedical Analysis, 115 (2015), 123., Hamase K, et al., Chromatography 39 (2018) 147-152).

The separative analysis system for optical isomers according to the invention may be a combination of multiple separative analysis methods. More specifically, the amount of a D-amino acid and/or L-amino acid in a sample can be measured using an optical isomer analysis method comprising a step of passing a sample containing a component with optical isomers through a first column filler as the stationary phase, together with a first liquid as the mobile phase, to separate the components in the sample, a step of separately holding each of the components in the sample in a multi loop unit, a step of passing each of the components in the sample that are separately held in the multi loop unit through a flow channel in a second column filler having an optically active center, as the stationary phase, together with a second liquid as the mobile phase, to separate the optical isomers among each of the sample components, and a step of detecting the optical isomers in each of the sample components (Japanese Patent Publication No. 4291628). In HPLC analysis, D- and L-amino acids are sometimes pre-derivatized with a fluorescent reagent such as o-phthalaldehyde (OPA) or 4-fluoro-7-nitro-2,1,3-benzoxadiazole (NBD-F), or diastereomerized using an agent such as N-tert-butyloxycarbonyl-L-cysteine (Boc-L-Cys) (Hamase, K. and Zaitsu, K., Bunseki Kagaku, Vol. 53, 677-690(2004)). Alternatively, the D-amino acids and/or L-amino acids may be measured by an immunological method using a monoclonal antibody that distinguishes optical isomers of amino acids, such as a monoclonal antibody that specifically binds to a D-amino acid or L-amino acid. When the total of D-amino acids and L-amino acids is to be used as the marker it is not necessary to separate the D-amino acids and L-amino acids, allowing the amino acids to be analyzed without separating the D-amino acids and L-amino acids. In such cases as well, separation and quantitation may be carried out using an enzyme method, antibody method, GC, CE or HPLC.

Throughout the present specification, the amounts of biomolecules such as D-amino acids, L-amino acids, creatinine and proteins, or drugs, may be expressed in any physical quantity that can be measured, which includes not only the simple mass, weight and amount of substance (mol), but also the mass, weight or amount of substance (mol) per tissue, cell, organ or molecular units or per volume or weight, or the mass, weight, amount of substance (mol), concentration, specific gravity or density in a biological sample such as feces, blood or urine.

As used herein, “indicator based on the amount of a D-amino acid” means the value of a measured amount of D-amino acid, or D-amino acid clearance, or D-amino acid excretion rate (NPL 5), or a formula or value corrected for purpose, using the amount of a D-amino acid as the explanatory variable, or a value calculated from the set formula. the value measured for a subject is the examination value of the indicator based on the amount of a D-amino acid. According to one aspect of the invention, the amount of the D-amino acid in the biological sample may be corrected using a physiological variable factor such as age, gender or BMI. When the dynamics of the D-amino acid are affected by kidney function, it may be corrected using a kidney function marker. Without intending to be limitative, the kidney function marker may be one or more selected from among creatinine, cystatin C, inulin clearance, creatinine clearance, urine protein, urine albumin, β2-MG, α1-MG, NAG, L-FABP, NGAL, glomerular filtration rate and estimated glomerular filtration rate (eGFR), with ratio of the amount of D-amino acid/the amount of creatinine as a specific example. Since D-amino acids in vivo are known to fluctuate in neurodegenerative diseases (such as ALS) and autoimmune disease (such as multiple sclerosis) (PTLs 1 to 2), they can be corrected by fluctuation factors and markers in different diseases.

As used herein, “validation of examination or diagnosis result” means validation of diagnosis result using an indicator based on a different principle, for a subject that has been diagnosed based on clinical testing which may include false positivity assessment, such as physician inquiry, or examination of a specimen harvested from a subject (such as biochemical examination, serologic examination, endocrine examination, tumor marker examination, microbiology examination, virology examination, gene/chromosome examination, cellular immunological examination or pathologic examination), image examination (such as endoscopy, contrast agent examination, ultrasonic examination, CT scan or MRI scan), gene panel examination, nematode examination, microRNA examination, AminoIndex^{R}, 5-ALA fluorescence risk examination, or examination relating to companion diagnostic for advance testing of specific drug effects or side-effects. As a specific example, a subject assessed to be positive by a given tumor marker may be assessed as true positive if assessed as positive by examination value with an indicator based on the amount of a D-amino acid in a biological sample, or false positive (type I error) if assessed as negative in the same.

The validation of examination or diagnosis result using an indicator based on the amount of a D-amino acid may be carried out using a determination value for the indicator (“reference range or “clinical decision value”, according to the invention). The determination value (reference range or clinical decision value) used for the invention is generally set at a 95% interval around the center of an examination value distribution for healthy subjects (reference individuals) who satisfy a given reference, or subjects with cancer, but any interval may be set according to the purpose. The determination value has a diagnosis threshold, treatment threshold or preventive medicine threshold based on a reference for assessing prognosis in regard to diagnosis, prevention or treatment for a given pathology. The threshold (cutoff value) may be set by a case control study, clinical medicine empirical rule, case series study, cohort study or expert consensus, using analysis data or results relating to predictability and assessability utilizing an ROC curve (Receiver Operating Characteristic curve), multivariate logistic regression model or Cox proportional hazard model. According to one embodiment, the validation results for cancer examination or diagnosis results for the subject can be provided by comparing the indicator with an determination value determined from the amounts of D-amino acids in biological samples from the subjects having cancer, for example.

As used herein, “classification of cancer progression” means classification of severity of cancer as a stage. Staging is mainly determined based on the TNM factors, i.e. tumor extent (T), degree of lymph node metastasis (N) and presence of other metastasis or sites (M), although clinical classification and pathological classification are also used. Clinical classification is carried out based on physical findings, image diagnosis, biopsy and cytodiagnosis, as a basis for deciding on the method of treatment. Pathological classification, on the other hand, is carried out based on material obtained from surgery or cytodiagnosis of peritoneal lavage, as a basis for evaluating prognosis. It is common to use the rules for handling various cancers as established by academic institutions or the TNM classification established by the Union For International Cancer Control, with classification in stages I to IV for stomach cancer, for example. According to one embodiment, the information relating to classification of cancer progression for a subject may be provided by comparing the indicator with an determination value determined from the amounts of a D-amino acid in biological samples from subjects with cancer whose cancer stage has been classified.

As used herein, “prediction of prognosis” means predicting and estimating the future course or outlook of the disease or treatment. Kaplan-Meier analysis, or cancer patient PaP score (Palliative Prognosis Score) or PPI (Palliative Prognostic Index) are typical prognosis prediction tools used to represent prediction of prognosis, in units of hours, days, weeks, months or years. Prognosis may be functional prognosis for an organ, life prognosis for estimated death, or tumor reduction, increase, metastasis or relapse, and it may be represented as variable evaluation parameters or units, the prediction of prognosis being important information for selecting treatment means. According to one embodiment, the information relating to prognosis prediction of cancer for a subject may be provided by comparing the indicator with an determination value determined from the amounts of D-amino acids in biological samples from subjects with cancer with information regarding prognosis.

As used herein, “selection of treatment means” refers to selection of optimal means for a subject diagnosed with a specific disease, such means being surgery, radiotherapy, chemotherapy, drug therapy, immunotherapy, alimentary therapy or exercise therapy, or other technologies (such as techniques or administration methods), or deciding on priority or taking additional preparation of the patient so as to optimize the treatment means. The criteria and purpose of selection may be curing of the disease, symptom reduction or elimination, halting or slowing of disease progression, prevention of the disease or symptoms, inhibiting aggravation of the underlying disease, avoiding or minimizing side-effects, or improving or maintaining cost effectiveness and QOL. According to one embodiment, information for selection of cancer treatment means for the subject may be provided by comparing the indicator with an determination value determined from the amounts of the D-amino acids in biological samples from subjects with cancer that responds and/or does not respond to cancer treatment means.

According to the invention, examination or diagnosis results can be validated using an indicator based on the amount of a D-amino acid in a biological sample of a subject with diagnosis of cancer or suspected cancer based on a clinical test. For an indicator based on the amount of a D-amino acid, as one aspect, different D-amino acid and L-amino acid profiles in biological samples from subjects with cancer and ones without can be utilized for assessment of true positivity and false positivity by comparing an examination value for the subject with an determination value for a previously established indicator based on the amount of D-amino acid (either a reference range or a clinical decision value). Tumor markers (such as SCC, CEA, CA19-9, AFP, PIVKA-II, Span-1 or anti-p53 antibody for gastrointestinal cancer) are substances produced by cancer cells or substances produced by reaction of patient cells with tumors, and their detection can be used for diagnosis of tumors or assessment of relapse, metastasis and therapeutic effects, but one problem is that healthy subjects and benign conditions can also exhibit positivity. Since intake, absorption, transport, distribution, metabolism (synthesis and decomposition), excretion and function of D-amino acids partially varies due to cancer, fluctuation of an examination value used as an indicator based on the amount of a D-amino acid in a biological sample differs in principle from fluctuation in conventional tumor markers, making the amount of D-amino acid in biological samples highly useful for assessment of true positivity and false positivity. When cancer is suspected based on subject symptoms but the results of a given examination have been negative, false negativity (type II error) or true negativity can be assessed using an indicator based on the amount of a D-amino acid. AminoIndex^{R} examination (NPLs 8 to 9) is based on the amounts of amino acids regardless of stereoisomerism, whereas gene-related examination is based on genotype, and therefore an indicator based on the amount of a D-amino acid which allows for examination of the phenotype of disease symptoms while distinguishing between D-amino acids and L-amino acids has different features from these examinations and can exhibit an effect of assessing true or false test results.

According to a different aspect, validation of examination and diagnosis results provided by the invention can be utilized for cancer screening or pathological diagnosis. According to yet another aspect, the results of validation of examination and diagnosis results may be used for screening of an effect, side-effect or secondary reaction in drug development, or assessment in a clinical trial or for an alternative endpoint. For validation of examination or diagnosis results, one or more D-amino acid types, L-amino acid types, correction factors and indicators may be used as a set of multiple indicators to be used simultaneously for a panel examination. The subject sample used may be the same as for examination, harvested at a different timing in relation to the response and properties against cancer. The subject may be a mammal such as a human, or an animal in which cancer has been induced by cancer cell graft, genetic modification or drugs, or it may be an individual, cells, tissue or organoid prepared as a prescribed cancer model.

According to the invention, an indicator based on the amount of a D-amino acid in a biological sample of a subject may be used to classify cancer progression (stage). According to one aspect, D-amino acid and L-amino acid profiles in a biological sample from a subject with cancer which vary according to cancer progression can be utilized to classify cancer progression by comparing an examination value as an indicator based on the amount of D-amino acid for the subject, with an determination value which have been previously established on the basis of the amounts of D-amino acid (either a reference range or a clinical decision value), thereby providing information relating to prognosis or treatment for the subject. For example, the information relating to classification of cancer progression for the subject may be provided by comparing the indicator with an determination value determined from the amounts of a D-amino acids in biological samples from patients with cancer whose cancer stage has been classified.

Since the present invention is carried out by comparing a specified determination value (reference range or clinical decision value) with an examination value for a subject, it is a preliminary diagnostic method or auxiliary diagnostic method intended to increase diagnosis precision for a physician based on validation results, without judgment by a physician. The method may be conducted by a non-physician such as a clinical tester, health examiner or data processing technician, or by an analysis system or program.

One embodiment of the invention provides information for assisting selection of treatment means for cancer using an indicator based on the amount of a D-amino acid in a biological sample of a subject. The difference in profiles of D-amino acids and L-amino acids in a biological sample of a subject, as response or prognosis of cancer after treatment, can be used to assist in selecting optimal means such as surgery, radiotherapy, chemotherapy, drug therapy, immunotherapy, alimentary therapy or exercise therapy, or determining priority for the same, depending on comparison of an determination value (reference range or clinical decision value) previously set for an indicator based on the amount of a D-amino acid with the examination value for the subject. Chemotherapy is treatment of cancer using an anticancer agent, which can provide a wider systemic effect compared to surgery or radiotherapy which have local effects. While surgical management (reduction surgery, routine surgery or expansion surgery) or endoscopic treatment are usually selected for early cancer and advanced cancer, chemotherapy is sometimes implemented in combination with surgery. Chemotherapy is implemented before surgery for the purpose of alleviating bleeding and burden on the body by shrinking the cancer, and after surgery for the purpose of inhibiting relapse, metastasis and proliferation of remaining cancer. Chemotherapy is also selected for unresectable cases, but according to another aspect, information can be provided to assist selection of optimal drugs or decision regarding prioritization, using an indicator based on the amount of a D-amino acid. As a specific example, the possibility of administering a drug may be determined by previously setting an determination value (reference range or clinical diagnosis value) for an indicator based on the amount of a D-amino acid, relating to an effect or side-effect of the drug or its secondary reactions, and comparing it with an examination value for the subject. According to another aspect, it is possible to provide information for predicting and assessing an effect or side-effect or secondary reaction after drug administration, depending on an indicator based on the amount of a D-amino acid, or for assisting a decision on continuing or halting administration or determining the dose or timing of administration. Drugs to be used include, but are not limited to, antineoplastic agents such as antimetabolites (fluorouracil (5-FU), tegafur-gimeracil-oteracil potassium combination (S-1), gemcitabine hydrochloride (GEM), levofolinate calcium (1-LV), folinate calcium (LV), tegafur-uracil combination, capecitabine or trifluridine-tipiracil hydrochloride combination), platinum formulations (cisplatin (CDDP), oxaliplatin, miriplatin hydrate), anthracyclines (epirubicin hydrochloride), topoisomerase inhibitors (irinotecan hydrochloride hydrate), microtubule inhibitors (paclitaxel, docetaxel hydrate), alkylating agents (streptozocin), molecular targeted drugs (anti-VEGF antibody formulations: bevacizumab, anti-EGFR antibody formulations: cetuximab, panitumumab, anti-HER2 antibody formulations: trastuzumab, anti-VEGFR antibody formulations: ramucirumab, BCR/ABL inhibitors: imatinib mesylate, multikinase inhibitors: sunitinib malate, regorafenib hydrate, sorafenib tosylate, lenvatinib mesylate, EGFR inhibitors: erlotinib hydrochloride, VEGF inhibitors: aflibercept beta and mTOR inhibitors: everolimus), immune checkpoint inhibitors (such as immune checkpoint molecule inhibitors selected from the group consisting of CTLA-4, PD-1, PD-L1, PD-L2, LAG-3, TIM3, BTLA, B7H3, B7H4, 2B4, CD160, A2aR, KIR, VISTA and TIGIT, for example, anti-PD-1 antibodies (nivolumab and pembrolizumab)), antitumor antibiotics (mitomycin C), adrenal cortical steroids (prednisolone, budesonide), and Chinese herbal medicines. Typical regimens for stomach cancer include SP therapy, XP therapy, SOX therapy and CapeOX (XELOX), with molecular targeted drugs being used in combination with microtubule inhibitors, topoisomerase inhibitors or immune checkpoint inhibitors depending on the therapeutic effect, based on the results of genetic testing or protein examination (such as HER2) of a subject, and the present invention can provide information necessary for their selection. For example, when an examination value for an indicator based on the amount of a D-amino acid is in a range judged suitable for use of an immune checkpoint inhibitor, it is possible to provide information necessary for selection, prioritization and switching of administration timing of the immune checkpoint inhibitor, in the context of a treatment policy for an enhanced therapeutic effect.

According to the invention, by adjusting the amount of the D-amino acid in vivo based on an examination value for an indicator based on the amount of a D-amino acid in a biological sample of a subject (for example, by adjusting the amount of the D-amino acid in a biological sample of the subject (such as blood) so that the value of an indicator based on the amount of the D-amino acid in the biological sample of the subject (such as blood) is within or near a prescribed range), it is possible to control progression of cancer, or to alter the response to treatment in the subject diagnosed with cancer and assist in selection of treatment means. For cancer prognosis during administration of a drug, the difference in profiles of D-amino acids and L-amino acids in a biological sample of the subject may be used to set beforehand a range or determination value (clinical decision value) for an indicator based on the amount of a D-amino acid which is expected as an effect of treatment, taking measures so that the examination value for the subject falls within that range (examination value adjustment). Specifically, when an examination value is within a set range by controlling the amount of a D-amino acid in vivo of a subject the value is adjusted so that it is maintained, when the examination value exceeds the set range the value is adjusted so that it is reduced, and when the examination value falls below the set range the value is adjusted so that it increases. According to another aspect, when a range or determination value (clinical decision value) for an indicator based on the amount of a D-amino acid has been set that produces a side-effect or secondary reaction, measures are taken so that the examination value of the subject is outside of that range. A drug or food that can raise or lower the amount of a D-amino acid in tissues, cells, organs or body fluids by external administration of the D-amino acids, or by addition or removal of the D-amino acids to or from a food (alteration of composition) may be used to adjust an examination value as an indicator based on the amount of a D-amino acid in a biological sample to be used for the invention. For example, drinking a water-soluble solution containing D-amino acids can increase the D-amino acid concentration in blood or tissues (NPL 1), while ingesting foods with the D-amino acids removed can lower D-amino acid concentrations in blood. The D-amino acids to be used may also contain D-amino acid modified forms or derivatives or pharmaceutically acceptable salts of the same, so long as they can raise or lower the amount of a D-amino acid in vivo to adjust examination values, and may also include pharmacologically acceptable carriers, diluents or excipients, or may be prepared as prodrugs. When a drug is used to adjust the examination value for a subject, it may be formulated with selection of the dosage form as appropriate for the desired route of administration. The dosage form may be designed as a tablet, capsule, liquid drug, powdered drug, granules or a chewable agent for use in oral administration, or as an injection, powdered drug or infusion preparation for parenteral administration. These formulations may also include various types of adjuvants such as carriers or other auxiliary agents that are used in drugs, including stabilizers, antiseptic agents, soothing agents, flavorings, taste correctives, aromatics, emulsifiers, fillers and pH adjustors, in ranges that do not interfere with the effect of the invention. The optical purity of the drug and its D-amino acid starting materials is preferably 50% or greater and more preferably 90% or greater, but the optical purity is not restricted and may be selected as desired within a range that exhibits an effect. The examination value for an indicator based on the amount of a D-amino acid may also be adjusted using an arbitrary physiological mechanism. As a specific example, the levels of D-amino acids can be controlled by activating a mechanism such as regulating (promoting or inhibiting) expression and/or activity (action, inhibition or stimulation) of proteins related to absorption, transport, distribution, metabolism (synthesis and/or decomposition), excretion or action of D-amino acids, or of D-amino acid transporters or receptors. The control agent for the amount of D-amino acid to be used for the invention may therefore be one that directly or indirectly promotes gene expression of a protein related to absorption, transport, distribution, metabolism or excretion of a D-amino acid, and for example, it may be a protein or a vector that expresses it, or it may be a factor that regulates activity upstream in a cascade that promotes expression of the protein, or a vector that expresses the factor. The control agent for the amount of D-amino acids to be used for the invention may also be, for example, one that directly or indirectly inhibits gene expression of a protein related to absorption, transport, distribution, metabolism or excretion of a D-amino acid, such as one selected from among low molecular compounds, aptamers, antibodies or antibody fragments, or antisense RNA or DNA molecules, RNAi-inducible nucleic acid, microRNA (miRNA), ribozymes or genome editing nucleic acids, as well as expression vectors for the same. It may be a protein related to absorption, transport, distribution, metabolism (synthesis and/or decomposition), excretion or action of a D-amino acid, for example, an enzyme such as D-amino acid oxidase (DAO), D-aspartate oxidase (DDO), serine isomerase (SRR) or DPP-4. As a specific example, a DAO inhibitor (such as Risperidone) inhibits oxidation of D-amino acids, increasing the amount of a D-amino acid, and it can therefore be used as an agent for controlling the amount of a D-amino acid. Since a D-amino acid transporter raises or lowers the amount of a D-amino acid at the transport source and transport destination, an agent that directly or indirectly acts on D-amino acid transporters may also be used for the invention. NPL 4 discloses that agonist/inhibitor D-amino acid transporter proteins such as the SMCT family or ASCT family which are expressed in the brain, kidneys and intestinal tract affect local levels of D-amino acids. These transporters are affected by coordination or competition via cotransport substances (such as sodium ions) or scaffolds, with D-amino acid transport activity also being controlled by sodium/glucose symporter (SGLT2) inhibitors, for example, and therefore agents that act on such transporters can be used as agents for controlling the amount of a D-amino acid. PTL 3 discloses that angiotensin 2 receptor blocker (ARB) alters D-amino acid levels in blood, and agents that act on such receptors can also be used as agents for controlling the amount of a D-amino acid. Since D-serine, D-alanine and glycine are co-agonists for NMDA-type glutamate receptors (NMDA receptor: N-methyl-D-aspartate receptor), NMDA receptor antagonists (such as memantine, ketamine, dextromethorphan, dextrorphan, amantadine, eliprodil, ifenprodil, phencyclidine, MK-801, dizocilpine, CCPene and flupirtine, or their pharmaceutically acceptable salts) may also be used to affect active amounts and activity of D-amino acids in vivo, and may be used as agents for controlling the amount of a D-amino acid. Preferred NMDA receptor antagonists for use according to the invention are memantine and its pharmaceutically acceptable salts. Drugs that exhibit effects via delta glutamate receptors and AMPA-type glutamate receptors may likewise be used for the invention.

Throughout the present specification, “agent for controlling the amount of a D-amino acid” refers to an agent that raises or lowers the amount of D-amino acid levels in vivo (such as cells, tissue, organs or body fluids) of a subject, or in isolated cells or tissue organoids by its application (such as administration), and it may act by any mechanism such as absorption, transport, distribution, metabolism (synthesis and/or decomposition) or excretion. In the case of a target D-amino acid level or concentration, the amount of the D-amino acid in the specimen may be evaluated by appropriate examination or monitoring.

As used herein, “aptamer” refers to a synthetic DNA or RNA molecule or peptide molecule that has the ability to specifically bind to a target substance, and it can be chemically synthesized rapidly in vitro. An aptamer used for the invention binds to a protein related to absorption, transport, distribution, metabolism or excretion of D-amino acids, thereby inhibiting its activity. The aptamer to be used for the invention can be obtained, for example, by selection by repetitive in vitro binding to various molecular targets such as small molecules, proteins and nucleic acids, using the SELEX method (see Tuerk C., Gold L., Science, 1990, 249(4968), 505-510; Ellington A D, Szostak J W., Nature, 1990, 346(6287):818-822; U.S. Pat. Nos. 6,867,289; 5,567,588; and 6,699,843).

As used herein, “antibody fragment” refers to a part of a full length antibody that maintains the activity of binding with antigen, and the concept generally includes the antigen-binding domain or variable domain. Examples of antibody fragments include F(ab′)2, Fab′, Fab and Fv antibody fragments (including scFv antibody fragments). The concept of antibody fragment also includes a fragment that is treated with a protease enzyme, often being reduced. The antibody or antibody fragment used for the invention may be any antibody such as a human-derived antibody, mouse-derived antibody, rat-derived antibody, rabbit-derived antibody, Camelidae (such as llama)-derived antibody or goat-derived antibody, and it may also be a polyclonal or monoclonal antibody, or a complete or shortened antibody (for example, a F(ab′)2, Fab′, Fab or Fv fragment), or a chimeric antibody, humanized antibody or fully human antibody.

As used herein, an “antisense RNA or DNA molecule” is a molecule having a nucleotide sequence complementary to functional RNA (sense RNA), such as messenger RNA (mRNA), and that forms a double strand with the sense RNA, having the function of inhibiting synthesis of the protein that is normally carried out by the sense RNA. According to the invention, an antisense oligonucleotide containing an antisense RNA or DNA molecule binds with mRNA of a protein related to absorption, transport, distribution, metabolism or excretion of D-amino acids, thereby inhibiting its translation to protein. This can reduce the expression level of the protein related to absorption, transport, distribution, metabolism or excretion of D-amino acids, thereby inhibiting its activity. The method of synthesizing the antisense RNA or DNA molecule for the invention may be any method known in the technical field.

As used herein, “RNAi-inducible nucleic acid” refers to a polynucleotide that is capable of inducing RNA interference (RNAi) by being introduced into cells, and it may usually be RNA or DNA, or a chimeric molecule of RNA and DNA, comprising 19 to 30 nucleotides, preferably 19 to 25 nucleotides and more preferably 19 to 23 nucleotides, optionally with desired modification. The RNAi may be produced on the mRNA, or on transcribed RNA just before processing, i.e. RNA having a nucleotide sequence including the exon, intron, 3′-untranslated region and 5′-untranslated region. The RNAi method that may be used for the invention may be induction of RNAi by a method such as (1) directly introducing short double-stranded RNA (siRNA) into cells, (2) incorporating short hairpin RNA (shRNA) into different expression vectors and introducing the vectors into cells, or (3) creating a vector that expresses siRNA by inserting short double-stranded DNA corresponding to the siRNA, between promoters in a vector having two promoters running in opposite directions, and introducing the vector into cells. The RNAi-inducible nucleic acid may include siRNA, shRNA or miRNA capable of cleaving D-serine transporter protein RNA or suppressing its function, and such RNAi nucleic acid may be directly introduced using liposomes or the like, or it may be introduced using an expression vector that induces the RNAi nucleic acid.

The RNAi-inducible nucleic acid for a protein related to absorption, transport, distribution, metabolism or excretion of D-amino acids to be used for the invention may be nucleic acid that exhibits a biological effect of inhibiting or significantly suppressing expression of the protein related to absorption, transport, distribution, metabolism or excretion of D-amino acids, and it can be synthesized by a person skilled in the art by referring to the nucleotide sequence of the protein. For example, it may be chemically synthesized using a DNA (/RNA) automatic synthesizer utilizing DNA synthesis technology such as the solid phase phosphoramidite method, or it may be synthesized by consignment to an siRNA-related contracted synthesis company (such as Life Technologies). According to an embodiment, the siRNA to be used for the invention may be one derived from short-hairpin-type double stranded RNA (shRNA) as the precursor, via processing with a dicer, which may be an intracellular RNase.

As used herein, “microRNA (miRNA)” is a single-stranded RNA molecule with a length of 21 to 25 bases, which contributes to regulation of post-transcriptional expression of genes in eukaryotes. Such miRNA generally recognizes 3′UTR in mRNA, inhibiting translation of target mRNA and inhibiting protein production. Thus, miRNA that can directly and/or indirectly lower expression levels of a D-serine transporter protein is also within the scope of the present invention.

As used herein, “ribozyme” is a general term for enzymatic RNA molecules that can catalyze specific cleavage of RNA. Ribozymes include large ones of 400 or more nucleotides such as M1 RNA, which are included in group I introns or RNase P, but some have active domains of about 40 nucleotides, known as hammerhead types or hairpin types (see Koizumi, M. and Ohtsuka, E., Tanpakushitsu, Kakusan, Kouso, 1990, 35, 2191, for example). For example, the self-cleaving domain of hammerhead ribozyme cleaves the 3′-end of C15 in the sequence G13U14C15, with formation of a base pair between U14 and A9 being considered important for activity, and potential cleavage at A15 or U15 instead of C15 (see Koizumi, M. et al., FEBS Lett, 1988, 228, 228, for example). If a ribozyme is designed with the substrate binding site being complementary to the RNA sequence near the target site, then it is possible to obtain a restriction enzyme RNA-cleaving ribozyme that recognizes the sequence UC, UU or UA in target RNA, and this can be produced by a person skilled in the art with reference to the following publications: Koizumi, M. et al., FEBS Lett, 1988, 239, 285; Koizumi, M and Ohtsuka, E. Tanpakushitsu, Kakusan, Kouso, 1990, 35, 2191; and Koizumi, M. et al., Nucl. Acids Res., 1989, 17, 7059. A hairpin ribozyme may also be used for the invention. This type of ribozyme is found, for example, on the minus strand of satellite RNA of tobacco ringspot virus (Buzayan, J M., Nature, 1986, 323, 349). It has been demonstrated that a target-specific RNA-cleaving ribozyme can be created from a hairpin ribozyme as well (see Kikuchi, Y. & Sasaki, N., Nucl. Acids. Res., 1991, 19, 6751; and Kikuchi, Y., Kagaku to Seibutsu, 1992, 30, 112, for example). By using a ribozyme to specifically cleave the transcription product of a gene coding for a D-serine transporter protein, it is possible to inhibit expression of the D-serine transporter protein.

As used herein, “genome editing nucleic acid” refers to a nucleic acid used for editing of a desired gene in a system utilizing a nuclease that is used for gene targeting. Nucleases used for gene targeting include known nucleases, and also novel nucleases to be used for future gene targeting. For example, known nucleases include CRISPR/Cas9 (Ran, F. A., et al., Cell, 2013, 154, 1380-1389), TALEN (Mahfouz, M., et al., PNAS, 2011, 108, 2623-2628) and ZFN (Urnov, F., et al., Nature, 2005, 435, 646-651).

Utilizing the fact that symbiotic bacteria such as enterobacteria are a source of D-amino acids, the microbiome or growth environment may be altered by means such as administration of antibiotics, intestinal regulators or oligosaccharides, or using probiotics, microbial transplant, fecal transplant or improvement of dysbiosis, thus making it possible to raise or lower the amount of a D-amino acid in vivo. Without being limitative, one example of probiotics is intake of yogurt containing 1073R-1 lactic acid bacteria, which is known to increase D-serine and decrease D-lysine in the stool, and such lactic acid bacteria may also be used as an agent for controlling the amount of a D-amino acid according to the invention.

A drug or food that can adjust an examination value as an indicator based on the amount of a D-amino acid, regardless of the mechanism, can be used as means for controlling the amount of the D-amino acid in vivo according to the invention.

Throughout the present specification, the term “drug” is used to include drugs and quasi drugs.

Throughout the present specification, the term “food” means food in general, but in addition to common foods including health foods, it also includes health functional foods such as specified health foods and nutritional function foods, as well as dietary supplements (supplements and nutritional supplements), feeds and food additives.

Another aspect of the invention provides a system or program that carries out the method for providing information associated with cancer for a subject. For example, the invention provides a system for providing information associated with cancer in a subject, comprising a memory unit, an input unit, an analytical measurement unit, a data processing unit and an output unit, wherein:
the memory unit stores the amount of a D-amino acid in a biological sample (such as blood, urine or feces) and a determination value associated with cancer, the determination value being selected from the group consisting of:
an determination value relating to the validity of cancer examination results or cancer diagnosis results determined from the amounts of D-amino acids in biological samples of patients with cancer;
an determination value relating to cancer progression determined from the amounts of the D-amino acids in the biological samples of the patients with cancer;
an determination value relating to cancer prognosis determined from the amounts of the D-amino acids in the biological samples of the patients with cancer; and
an determination value for selection of cancer treatment means determined from the amounts of the D-amino acids in the biological samples of the patients with cancer;
the analytical measurement unit separates and quantifies the D-amino acid in the biological sample of the subject;
the data processing unit selects information associated with cancer in the subject by comparing an indicator based on the amount of the D-amino acid of the subject with the determination value stored in the memory unit; and
the output unit outputs the information.

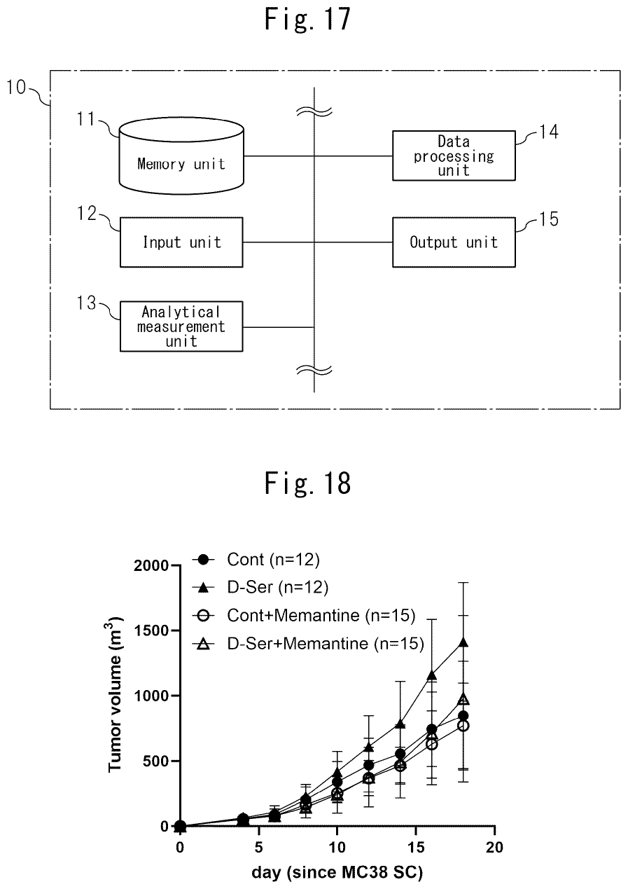

FIG. 17 is a block diagram of a system according to the invention. The sample analysis system 10 shown in FIG. 17 is constructed so as to allow the method of the invention to be carried out. The sample analysis system 10 comprises a memory unit 11, an input unit 12, an analytical measurement unit 13, a data processing unit 14 and an output unit 15, and allows analysis of biological samples and output of information regarding cancer in a subject.

More specifically, the memory unit 11 in the sample analysis system 10 of the invention may store the amount of a D-amino acid in a biological sample inputted through the input unit 12, and a determination value associated with cancer, the analytical measurement unit 13 may isolate and quantify the biological sample, the data processing unit 14 may compare an indicator based on the amount of the D-amino acid of the subject with the determination value stored in the memory unit to select information associated with the cancer of the subject, and the output unit 15 may output the information.

The memory unit 11 has a portable storage device which may be a memory device such as a RAM, ROM or flash memory, a fixed disk device such as a hard disk drive, or a flexible disk or optical disk. The memory unit stores data measured by the analytical measurement unit, data and instructions inputted from the input unit, and results of computation processing by the data processing unit, as well as the computer program and database to be used for processing by the information processing equipment. The computer program may be a computer readable recording medium such as a CD-ROM or DVD-ROM, or it may be installed via the internet. The computer program is installed in the memory unit using a commonly known setup program, for example. The memory unit stores data for a determination value associated with cancer previously inputted through the input unit 12.

The input unit 12 is an interface and also includes operating devices such as a keyboard and mouse. This allows the input unit to input data measured by the analytical measurement unit 13 and instructions for computation processing to be carried out by the data processing unit 14. When the analytical measurement unit 13 is external, for example, the input unit 12 may also include an interface unit allowing input of measured data through a network or storage medium, separately from the operating device.

The analytical measurement unit 13 measures at least the amount of a D-amino acid of a biological sample. The analytical measurement unit 13 may therefore have a construction allowing separation and measurement of the D-forms and L-forms of amino acids. The amino acids may be analyzed one at a time, or some or all of the amino acid types may be analyzed at once. With no intention to be limitative, the analytical measurement unit 13 may be a chiral chromatography system comprising a sample introduction inlet, an optical resolution column and a detector, for example, and it is preferably a high-performance liquid chromatography system. From the viewpoint of detecting the amounts of only specific amino acids, quantitation may be carried out by an enzyme method or immunological method. The analytical measurement unit 13 may be constructed separately from the system for evaluating kidney pathology, and measured data may be inputted through the input unit 12 using a network or storage medium.

The data processing unit 14 compares an indicator based on a measured amount of D-amino acid with an determination value stored in the memory unit, allowing information associated with cancer of a subject to be selected. The indicator based on the amount of a D-amino acid may be a formula or value obtained by correcting with the amount of a substance in vivo of the subject (for example, the amount of an L-amino acid or a kidney function marker), or it may be a formula or value obtained by correcting with a physiological variable factor such as age, gender or BMI.

The data processing unit 14 carries out various computation processing operations on the data measured by the analytical measurement unit 13 and stored in the memory unit 11, based on a program stored in the memory unit. The computation processing is carried out by a CPU in the data processing unit. The CPU includes a functional module that controls the analytical measurement unit 13, input unit 12, memory unit 11 and output unit 15, with the functional module performing various control operations. Each of the units may be constructed by independent integrated circuits, microprocessors and firmware.

The output unit 15 is constructed so as to output the information associated with cancer of the subject as the result of computation processing by the data processing unit. The output unit 15 may be output means such as a display device with a liquid crystal display that directly displays the computation processing results, or a printer, or it may be an interface unit for output to an external memory unit or output to a network.

According to another aspect, the invention may be a program that causes an information processor to carry out a method for providing information associated with cancer for a subject.

According to one embodiment, the invention may be a method for treating cancer in which a subject is treated by cancer treatment means selected based on information associated with cancer provided by an information device in which a method, system or program is installed. Referring to information associated with cancer provided by the invention allows optimal treatment means to be selected for the subject.

The content of all the patent and non-patent literature and references explicitly cited throughout the present specification are incorporated herein by reference.

The present invention will now be explained in detail by examples, with the understanding that these examples are in no way limitative on the invention. A person skilled in the art may easily implement modifications and changes to the invention based on the description in the present specification, and these are also encompassed within the technical scope of the invention.

### EXAMPLES

The following symbols are used throughout the Examples.
D-AA: D-amino acid
L-AA: L-Amino acid
Asn: Asparagine
Ser: Serine
Ala: Alanine
Pro: Proline
Leu: Leucine
PD-AA: D-amino acid concentration in plasma (nmol/mL)
PL-AA: L-amino acid concentration in plasma (nmol/mL)
PCre: Creatinine concentration in plasma
UD-AA/Cre: Amount of D-amino acid in urine/The amount of creatinine in urine (nmol/mg)
UL-AA/Cre: Amount of L-amino acid in urine/The amount of creatinine in urine (nmol/mg)
FD-AA: Amount of D-amino acid in feces (nmol/g)
FL-AA: Amount of L-amino acid in feces (nmol/g)

$$P \% D : PD ‐ AA / PD ‐ AA + PL ‐ AA \times 100 ( \% )$$

Subjects were either subjects with cancer diagnosed at Keio University Hospital or subjects without cancer, kidney or other disease diagnosis based on a complete medical checkup (healthy subjects: see M. Suzuki, et al, Amino Acids, 54, 421-432 (2022)), with plasma separated from blood collected from each subject after fasting for 2 hours or longer being provided for 2D-HPLC chiral amino acid analysis, and the acquired data being compared and analyzed. Both studies were approved by an ethics committee at Keio University Hospital, with written informed consent being obtained from both participants. The subjects with cancer were given standard treatments in accordance with the academic guidelines of Keio University Hospital.

### [Example 1] Assessment by Indicator Based on the Amount of D-Amino Acid in Blood

The test specimens were D-amino acids in plasma harvested from 25 individuals with stomach cancer, 6 individuals with esophageal cancer and 81 healthy individuals, none of which had used immune checkpoint inhibitors as antineoplastic agents, and indicators used for examination or diagnosis results validation and/or classification of cancer stage were analyzed. The determination value (cutoff value) or candidate determination value on the ROC curve was derived from the point of minimum distance between the curve and the point where the positive rate (sensitivity) was 1.00 (100%) and the specificity was 1.00 (100%).

(1) Assessment with “PD-AA” as Indicator

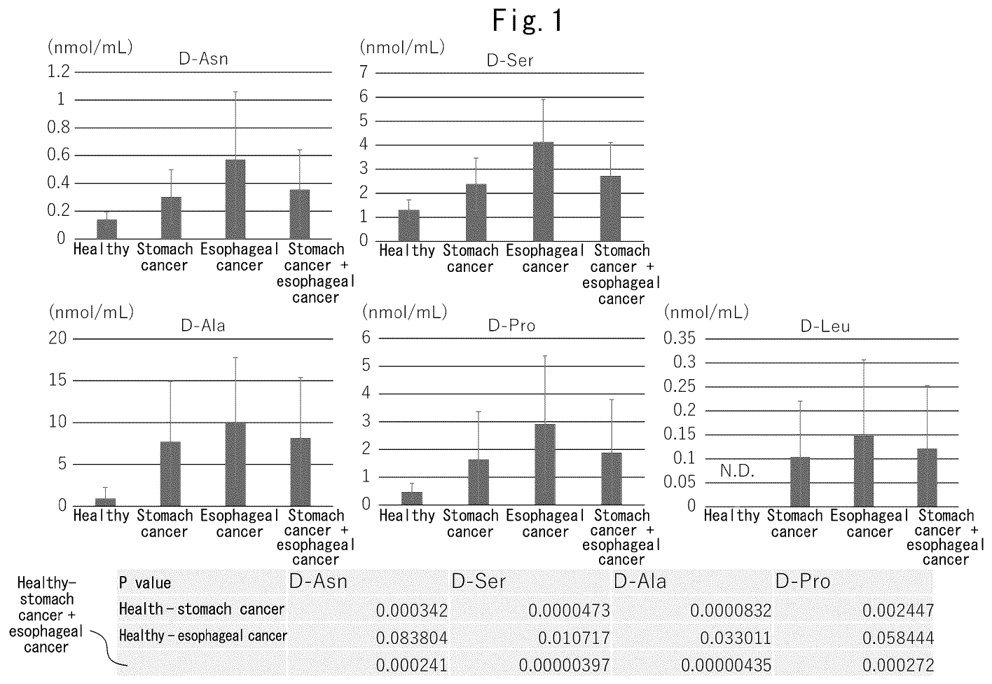

FIG. 1 shows amounts of D-Asn, D-Ser, D-Ala, D-Pro and D-Leu detected in plasma in the cancer subject group and the healthy subject group, as PD-AA (nmol/mL).

Since D-Leu is detected only in individuals with cancer while not being observed in the plasma of healthy individuals, it can be used for qualitative examination, and a positive rate (sensitivity) of 100% and a specificity of 48.4% were shown in this case.

For PD-Asn, PD-Ser, PD-Ala and PD-Pro, the results of a t test between the cancer subject group and the healthy group were as shown in the table at bottom. Since the PD-AA detected in plasma from cancer patients was significantly increased, the indicator using D-amino acids in blood allows validation of examination and diagnosis results using the “amount of D-amino acid in blood” represented by PD-AA.

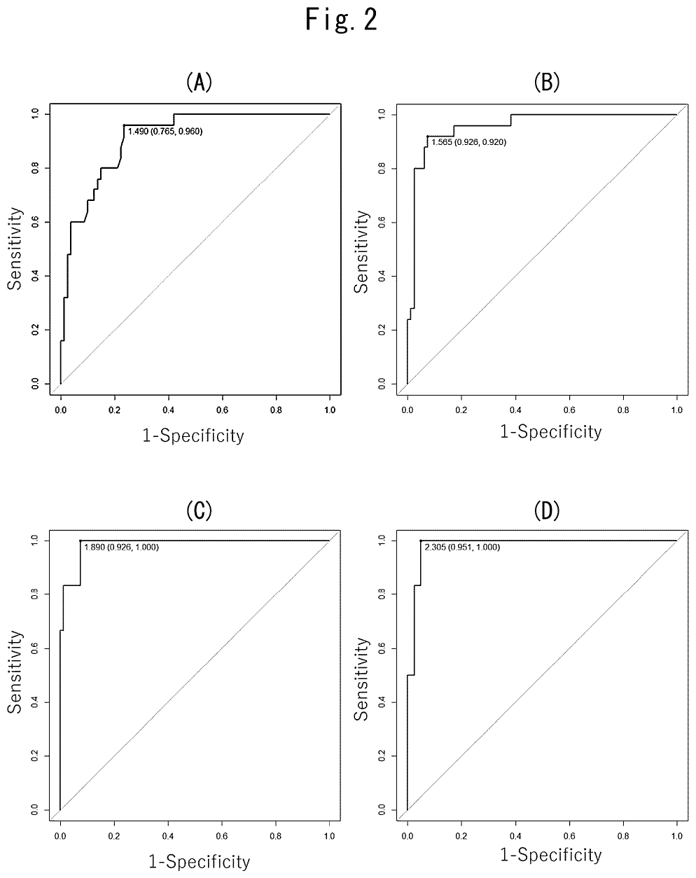

Based on analysis of the ROC curve, with the determination value for PD-Ser for stomach cancer set as 1.49, the positive rate was 96.0% and the specificity was 76.5% (FIG. 2A), and with the determination value for PD-Ala set as 1.57, the positive rate was 92.0% and the specificity was 92.6% (FIG. 2B). With the determination value for PD-Ser for esophageal cancer set as 1.89, the positive rate was 100% and the specificity was 92.6% (FIG. 2C), and with the determination value for PD-Ala set as 2.31, the positive rate was 100% and the specificity was 95.1% (FIG. 2D).

This allows mutual validation of examination/diagnosis results by a panel examination of PD-AA for multiple chiral amino acids in individual subjects with suspected cancer.

For esophageal cancer, an ROC curve from the regression equation:

$$2 \text{.03} \times D ‐ Ser + 0.268 \times D ‐ Ala ‐ 7.75$$

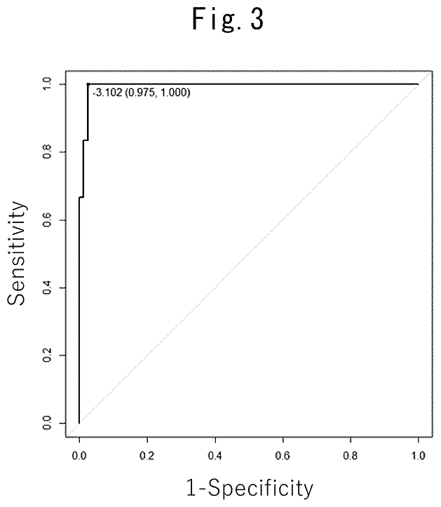

obtained from examination values for PD-Ser and PD-Ala was analyzed, and when the determination value calculated from the formula was set to −3.10, the positive rate was 100% and the specificity was 97.5% (FIG. 3).
(2) Assessment with “P % D” as Indicator

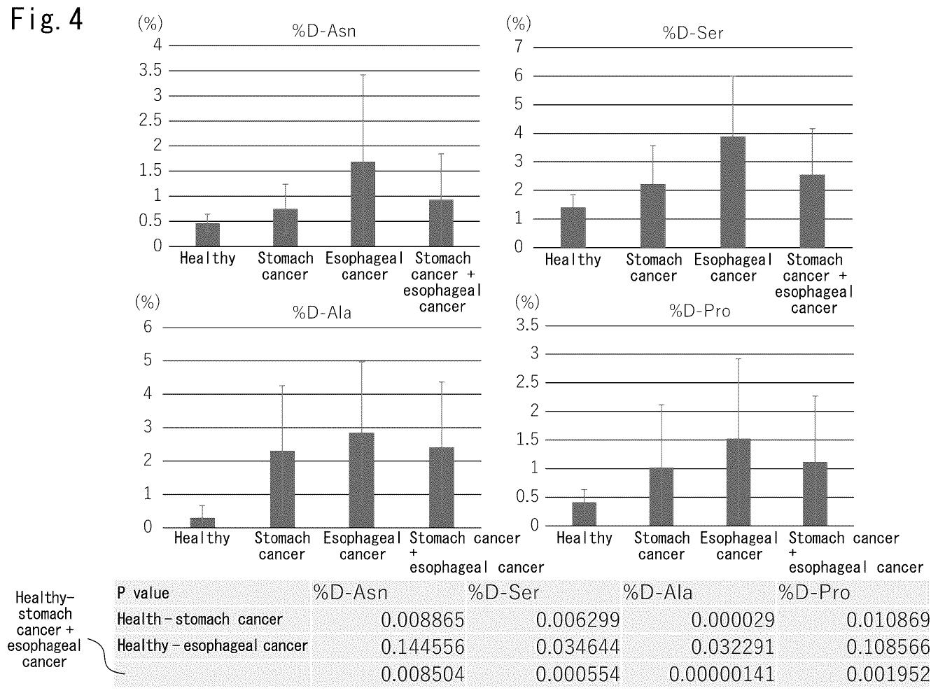

FIG. 4 shows amounts of Asn, Ser, Ala and Pro detected in plasma in the cancer subject group and the healthy subject group, as PD %.

For P % D-Asn, P % D-Ser, P % D-Ala and P % D-Pro, the results of a t test between the cancer subject group and the healthy group were as shown in the table at bottom. Since the P % D-AA detected in plasma from cancer patients was significantly increased, the indicator using D-amino acids in blood allows validation of examination and diagnosis results using the “ratio of the amount of D-amino acid and L-amino acid in blood” represented by P % D-AA.

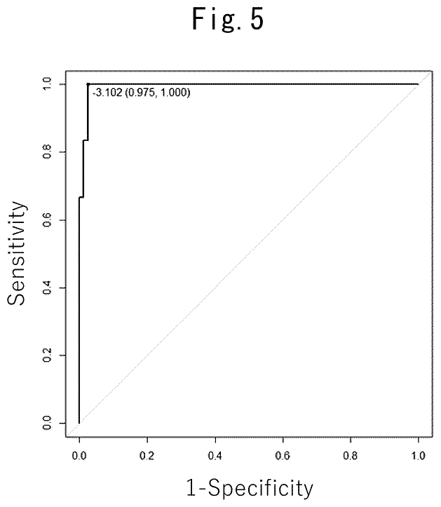

Based on analysis of the ROC curve, with the determination value for P % D-Ala for stomach cancer set as 54.6%, the positive rate was 88.0% and the specificity was 93.8% (FIG. 5).

This allows validation of mutual examination/diagnosis results by a panel examination of P % D for multiple chiral amino acids in individual subjects with cancer.

An ROC curve from the regression equation:

$$2.45 \times \% D ‐ Ala + 1.06 \times \% D ‐ Asn - 3.57$$

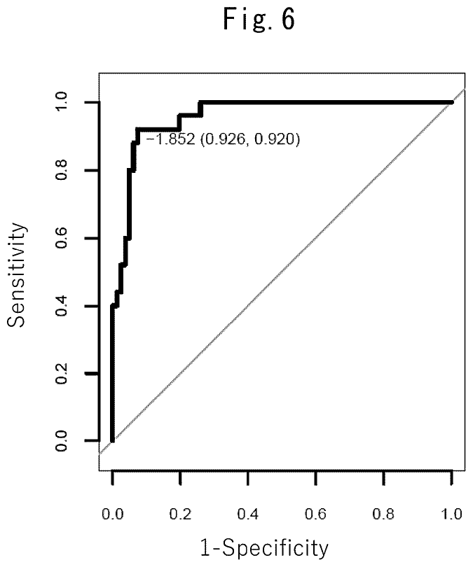

obtained from examination values for P % D-Ala and P % D-Asn was analyzed, and when the determination value calculated from the formula was set to −1.85, the positive rate was 92.0% and the specificity was 92.6% (FIG. 6).
(3) Assessment with “PD-AA/PCre” as Indicator

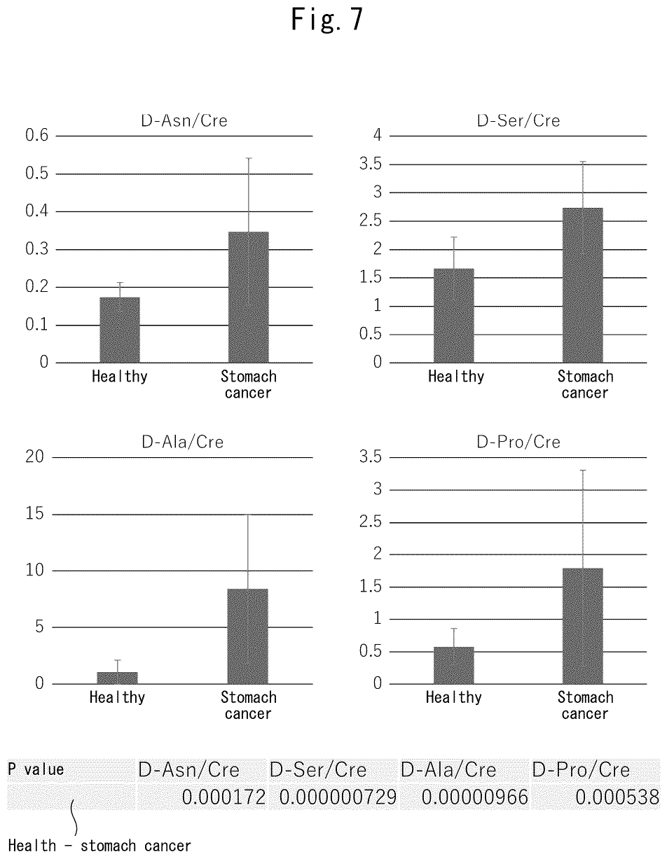

FIG. 7 shows PD-AA/PCre corrected with the kidney function marker Cre, for D-Asn, D-Ser, D-Ala and D-Pro detected in blood plasma of the cancer subject group and the healthy group.

For PD-Asn/PCre, PD-Ser/PCre, PD-Ala/PCre and PD-Pro/PCre, the results of a t test between the cancer subject group and the healthy group were as shown in the table at bottom. Since the PD-AA/PCre detected in plasma from cancer patients was significantly increased, the indicator using D-amino acids in blood allows validation of examination and diagnosis results using the “amount of D-amino acid in blood” represented by PD-AA.

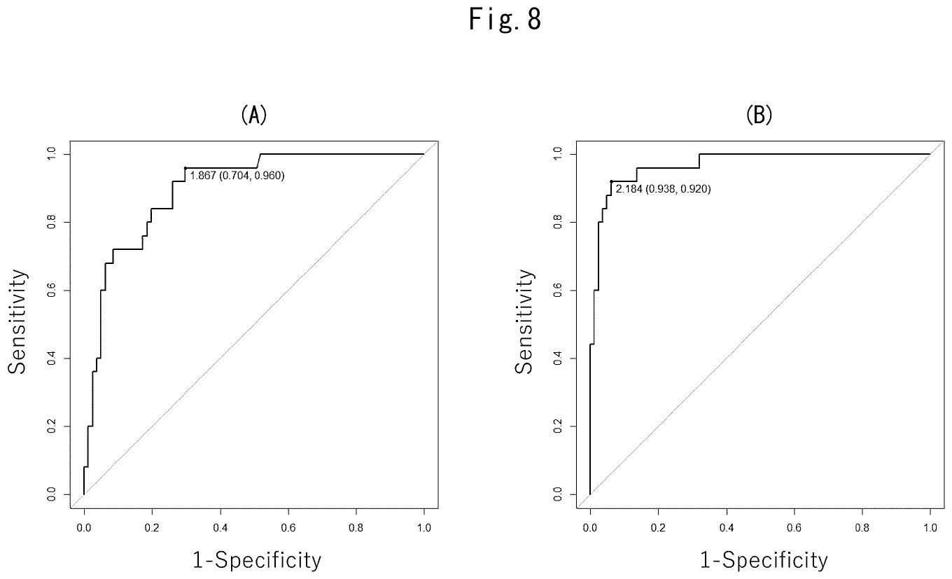

Based on analysis of the ROC curve, with the determination value for PD-Ser/PCre for stomach cancer set as 1.87, the positive rate was 96.0% and the specificity was 70.4% (FIG. 8A), and with the determination value for PD-Ala/PCre set as 2.18, the positive rate was 92.0% and the specificity was 93.8% (FIG. 8B).

This allows validation of mutual examination/diagnosis results by a panel examination of PD-AA for multiple chiral amino acids in individual subjects with suspected cancer.

An ROC curve from the regression equation:

$$0.983 \times D ‐ Ala / Cre + 1.96 \times D ‐ Ser / Cre ‐ 8.1$$

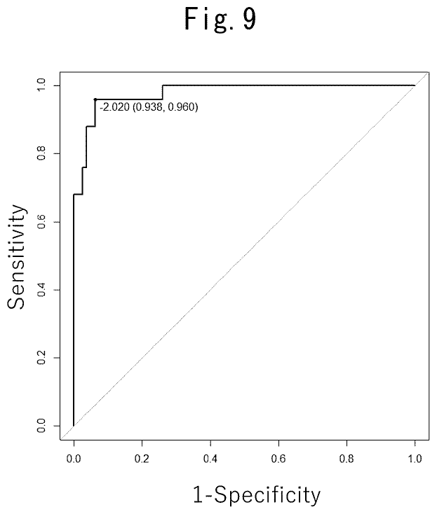

obtained from examination values for PD-Ser/Cre and PD-Ala/PCre was analyzed and when the determination value calculated from the formula was set to −2.02, the positive rate was 96.0% and the specificity was 93.8% (FIG. 9).
(4) Classification of Cancer Progression with “PD-AA” and “P % D-AA” as Indicators.

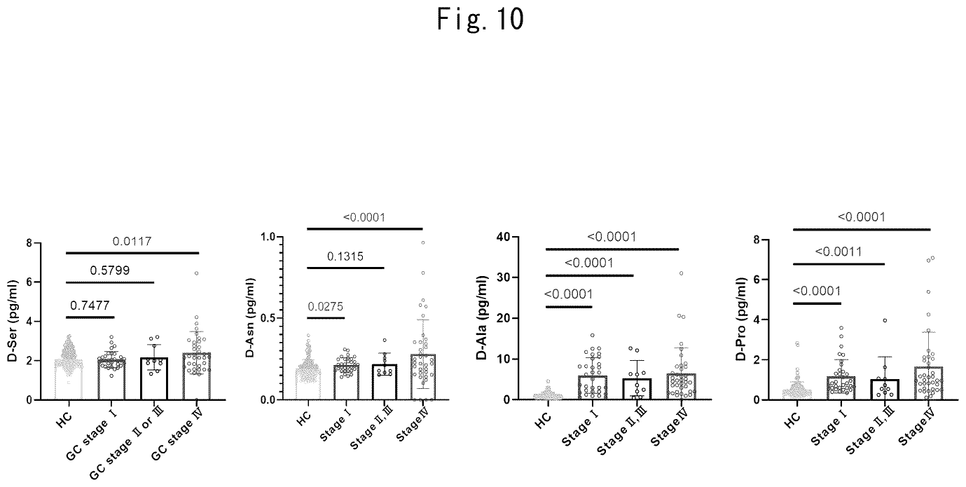

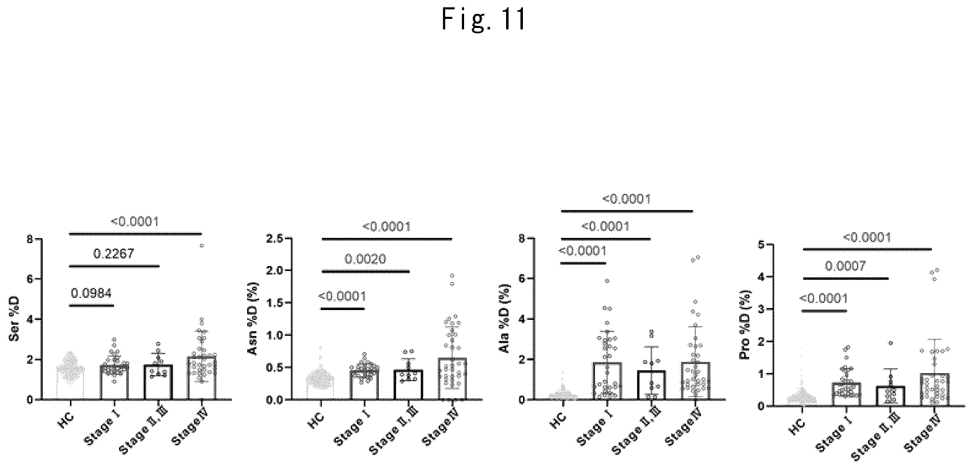

For PD-AA and P % D-AA in stomach cancer, classification of stage (Stage-I to IV) was according to the Guidelines for Stomach Cancer, 6th Edition (Japanese Gastric Cancer Association, July 2021), and a t test was conducted (FIGS. 10 and 11).

PD-AA and P % D-AA, as markers for D-Asn, D-Ala and D-Pro, were significantly increased in the Stage-I group compared to the healthy group, and were therefore useful for early validation of examination and diagnosis. This allows validation of mutual examination/diagnosis results and assessment of disease stage, by a panel examination of PD-AA for multiple chiral amino acids in individual subjects with suspected cancer.

When either or both PD-Ala and PD-Pro are increased, the progression may be classified as Stage-I to III, and when PD-Ser is simultaneously increased, it may be classified as Stage-IV. When one or more than one of P % D-Asn, P % D-Ala and P % D-Pro are increased, the progression may be classified as Stage-I to III, and when P % D-Ser is simultaneously increased, it may be classified as Stage-IV.

### [Example 2] Prognosis Prediction by Indicator Based on Amount of D-Amino Acid in Blood

The test specimens were analyzed for the indicators used for prediction of prognosis based on the amounts of D-amino acids in blood plasma harvested at the start of drug treatment from 28 subjects with unresectable relapsed stomach cancer, who had been administered the anti-PD-1 antibody formulation nivolumab, which is an immune checkpoint inhibitor (ICI) (antineoplastic agent). Treatment and use of the immune checkpoint inhibitor was in accordance with the Guidelines for Stomach Cancer, 6th Edition (Japanese Gastric Cancer Association, July, 2021), by intravenous infusion of nivolumab administered 480 mg per day for 2 or 4 weeks, within the scope of insurance. According to the Guidelines for Stomach Cancer, patients with tumors that have high microsatellite instability (MSI-High) are, as an exception, allowed to use anti-PD-1 antibody from second-line treatment. The criteria for measuring effectiveness of cancer treatment, as evaluation for prognosis, is classified as complete response CR (all signs of cancer disappear), partial response PR (condition improved), stabilized SD (no change) or progressed PD (condition worsened). CR or PR with improvement in the condition for 6 months or longer was observed in 7 cases, SD with progression-free survival for less than 6 months was observed in 8 cases, and non-responsive PD was observed in 13 cases.

(1) Prognosis Prediction of SD or PD with “PD-AA” as Indicator

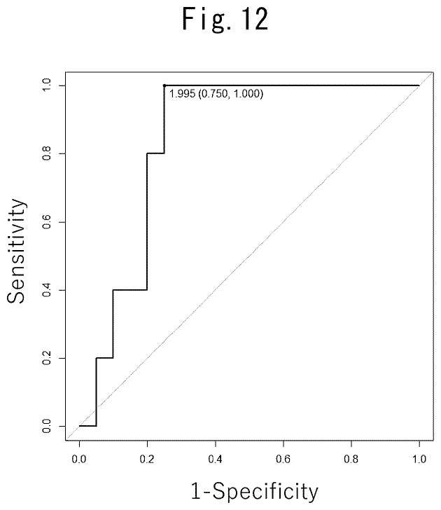

When the determination value for PD-Ser was 2.00 based on analysis of an ROC curve with outcome of CR-PR and SD-PD after nivolumab administration, the poor prognosis rate (sensitivity) was 100% and the specificity was 75.0% (FIG. 12).

This allows validation of mutual prognosis prediction by a panel examination of different indicators for multiple chiral amino acids in individual subjects with cancer.

Based on analysis of ROC curves for values calculated from a regression equation obtained from examination values for different indicators:
when the prognosis prediction value (determination value) was set to −1.24 by:

$$- 21.6 \times D ‐ Ser - 0.037 xL ‐ Ala + 46.7 ,$$

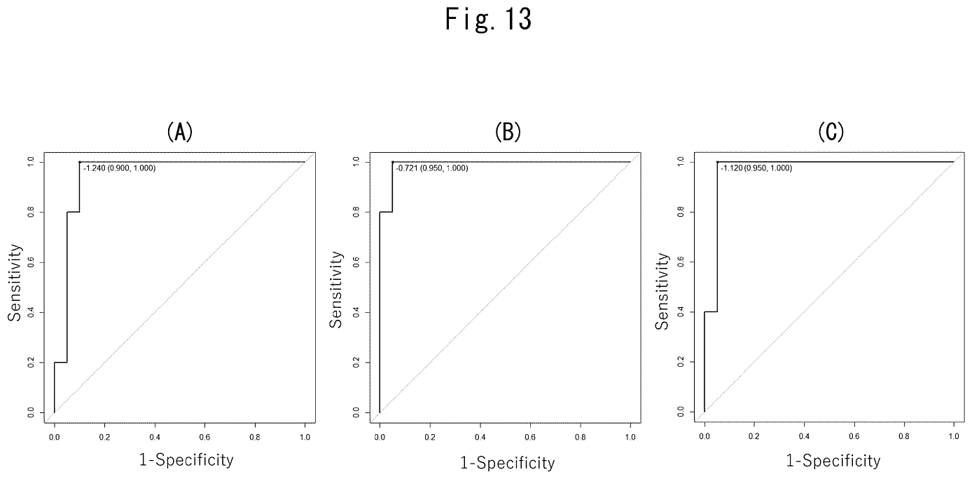

the SD-PD (poor prognosis rate) was 100% and the specificity was 90.0% (FIG. 13A),
when the prognosis prediction value (determination value) was set to −0.72 by:

$$- 8 7.4 \times D ‐ Ser - 0.151 \times 6.11 \times D ‐ Pro + 195.6 ,$$

the SD-PD (poor prognosis rate) was 100% and the specificity was 95.0% (FIG. 13B), and
when the prognosis prediction value was set to −1.12 by:

$$- 1 8. \times D ‐ Ser - 0.0332 \times L ‐ Ala + 3.47 \times D ‐ Asn / Cre + 38.5 ,$$

the poor prognosis rate was 100% and the specificity was 95.0% (FIG. 13C).

When the threshold calculated from 95% confidence interval for the PD-Ser examination value in the healthy group was set to 2.12, the SD-PD (poor prognosis rate) for subjects with higher indicators was 100% and the specificity was 75.0%.

This prognosis prediction can assist in selection of treatment means such as drug administration.

(2) Prognosis Prediction of PD with “PD-AA” as Indicator

With t test of PD-AA for the CR-SD group (non-PD group) and PD group after nivolumab administration, significantly high values were exhibited for PD-Ser (p=0.0021) and PD-Asn (p=0.0158).

When the threshold calculated from 95% confidence interval for the PD-Ser examination value in the healthy group was set to 2.12, the PD (poor prognosis rate) for subjects with higher indicators was 100% and the specificity was 75.0%.

This allows validation of mutual prognosis prediction by a panel examination of different indicators for multiple chiral amino acids (such as D-Ser and D-Ala) in individual subjects with cancer.

By analysis using the stepwise method and ROC curve, with the prediction formula for Nivolumab non-response (PD):

$$- 4 \text{.65} \times D ‐ Ser + 0 \text{.32} \times D ‐ Ala + 8.28$$

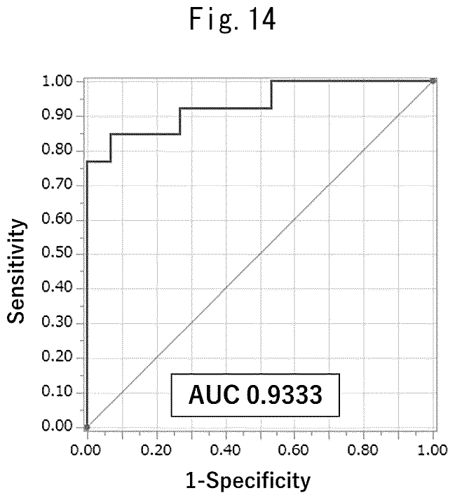

when the prognosis prediction value (determination value) was set to −9.01 (D-Ser 2.18, D-Ala 3.56), non-responsive cases were detected with PD (poor prognosis rate) of 76.9% and specificity of 100%. The Area under the curve (AUC) for this ROC curve was 0.9333 (FIG. 14).

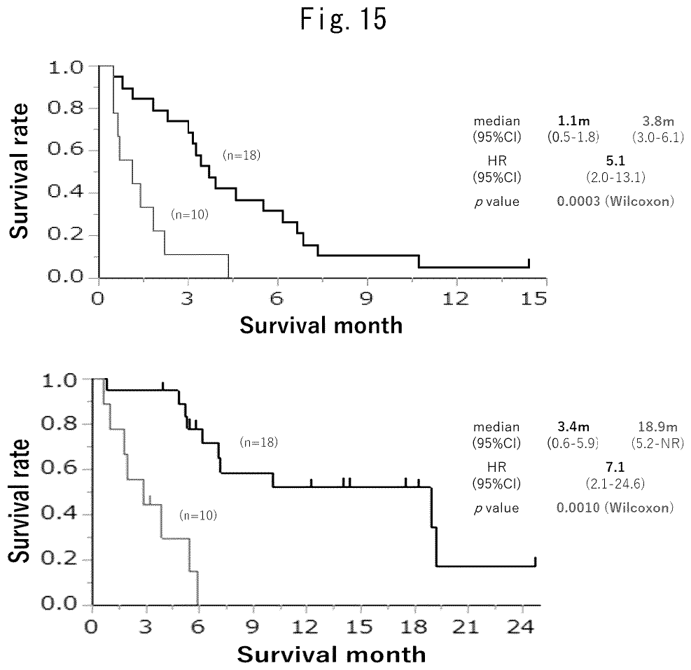

When the progression-free survival period or overall survival period was analyzed for 2 separate groups with determination value −9.01, the median was 1.1 months for progression-free survival (95% confidence interval: 0.5-1.8) vs 3.8 months (95% confidence interval: 3.0-6.1), with a hazard ratio of 5.1 (95% confidence interval: 2.0-13.1), p=0.0003, and the median was 3.4 months for overall survival (95% confidence interval 0.6-5.9) vs 18.9 months (95% confidence interval: 5.2—not reached), with a hazard ratio of 7.1 (2.1-24.6), p=0.001, all of which showed a significant difference (FIG. 15).

Such prognosis prediction can assist in selection of treatment means such as drug administration.

It is said that median overall survival for advanced unresectable relapsed stomach cancer patients undergoing third-line or later anticancer agent treatment is 6 months or less, and therefore when the selected anticancer agent is not effective there may be no major advantages from a health and economic standpoint. Predicting non-response or effectiveness of treatment and selecting suitable treatment methods (such as anticancer agents) is of considerable benefit for patients, and the use of appropriate and effective predictive markers can be especially effective in terms of improving QOL and alleviating the overall economic burden on the medical system.

### [Example 3] Prognosis by Controlling the Amount of D-Amino Acid In Vivo

Female Ly.5.1 mice (6- to 8-week-old) were given drinking water without D-amino acids (control group: n=11), with 1% D-Ser solution (D-Ser group: n=11) or with 1% D-Ala solution (D-Ala group: n=12), with the starting day as Day-14, MC38 cells (colorectal cancer line, 5×10⁵) were grafted after 2 weeks (Day-0), and after another 3 weeks (Day-20, 21) the tumors were measured for volume and weight, and provided for analysis.

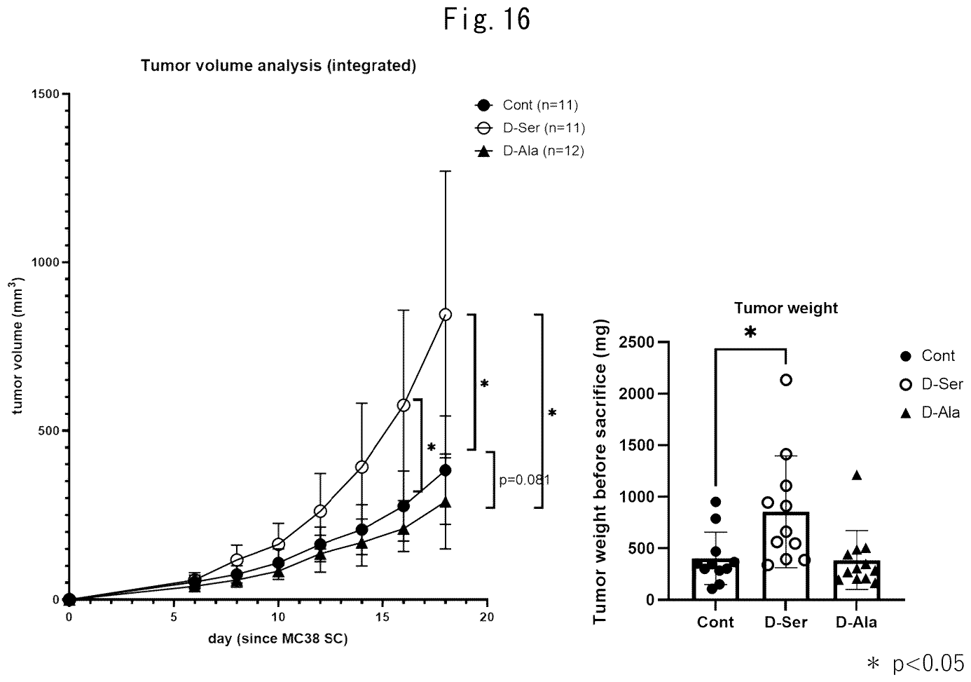

The D-Ser group had increased PD-Ser and significantly greater tumor volume and weight compared to the control group, based on which cancer prognosis was assessed to be poor (FIG. 16).

These data indicated that maintaining the amount of D-Ser in vivo, PD-Ser, at a low level can inhibit aggravation of cancer.

### [Example 4] Suppression of Tumor Increase by NMDA Receptor Antagonist

Female Ly.5.1 mice (6- to 8-week-old) were given drinking water without D-amino acids (control group) or with 1% D-Ser solution (D-Ser group), and were subcutaneously grafted with MC38 cells (colorectal cancer line, 5×10⁵), after which they were divided into a memantine-administered group and a control group, the administered group being given daily intraperitoneal administration of the NMDA receptor antagonist memantine (10 to 20 μg/BW (g)) from Day 4 onward. As a result, administration of memantine was found to inhibit the increase in tumor volume associated with D-Ser (FIG. 18).

This clearly demonstrated that lowering levels of D-amino acids acting on D-amino acid receptor proteins can exhibit an anticancer effect whereby tumor increase is inhibited.

### [Example 5] Detection and Stage Classification of Cancer by Indicator Based on the Amount of D-Amino Acid in Urine

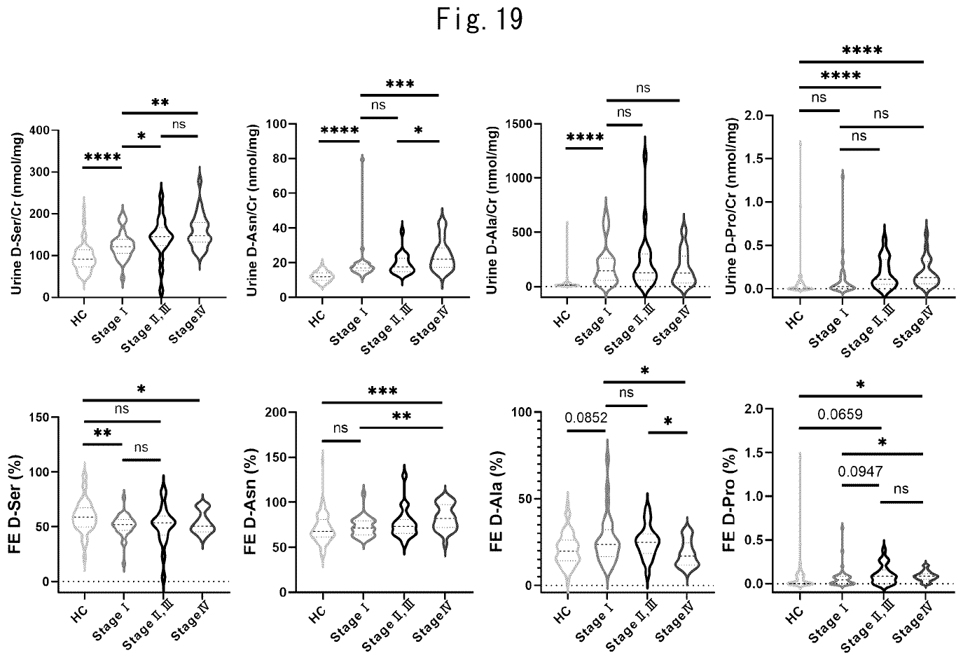

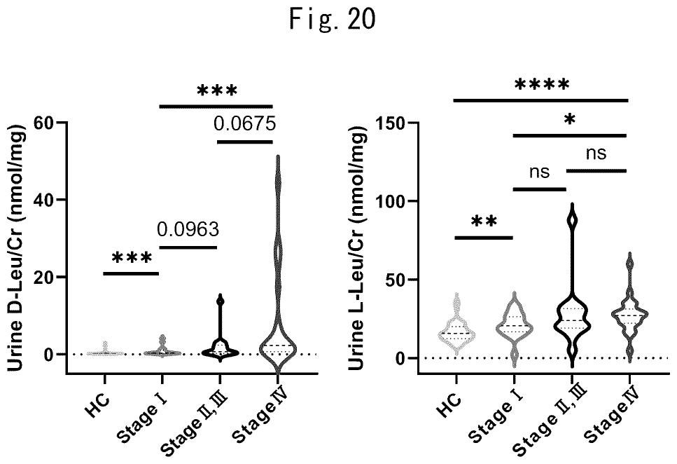

Patients undergoing complete medical checkup (71 healthy subjects) and 78 stomach cancer patients (Stage I: 33 individuals, Stage II, III: 20 individuals, Stage IV: 25 individuals) had urine specimens harvested after diagnosis and prior to treatment, the UD-AA was measured, and the UD-AA/Cre and excretion rates were calculated. The UD-AA/Cre ratio tended to increase similar to PD-AA, allowing provision of information relating to cancer detection and progression (FIG. 19 top, FIG. 20 left). UD-Leu/Cre, in particular, is not detected in most healthy subjects and can therefore be used for effective qualitative examination, allowing highly precise assessment by validation of cancer detection and diagnosis results, and classification of progression, based on panel examination using an indicator based on multiple UD-AA and PD-AA.

The excretion rate of D-Asn and D-Leu (FED-AA) also increases with progression of cancer, and information relating to cancer progression can thus be provided.

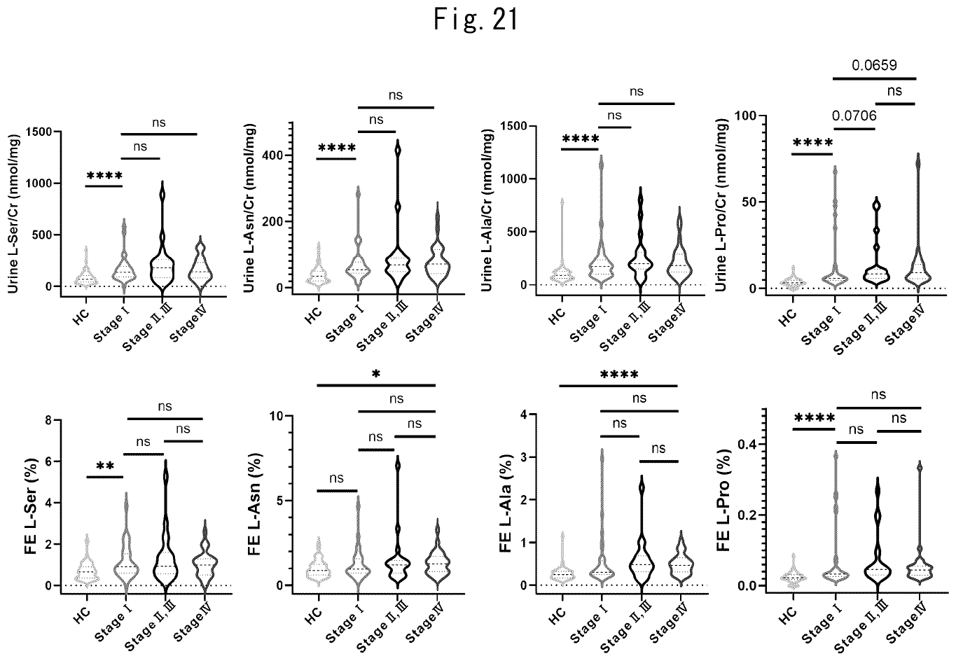

While UL-AA/Cre can be detected in Stage I cancer (FIG. 21), UD-AA/Cre can provide more precise information in regard to progression.

### [Example 6] Detection and Stage Classification of Cancer by Indicator Based on the Amount of D-Amino Acid in Feces

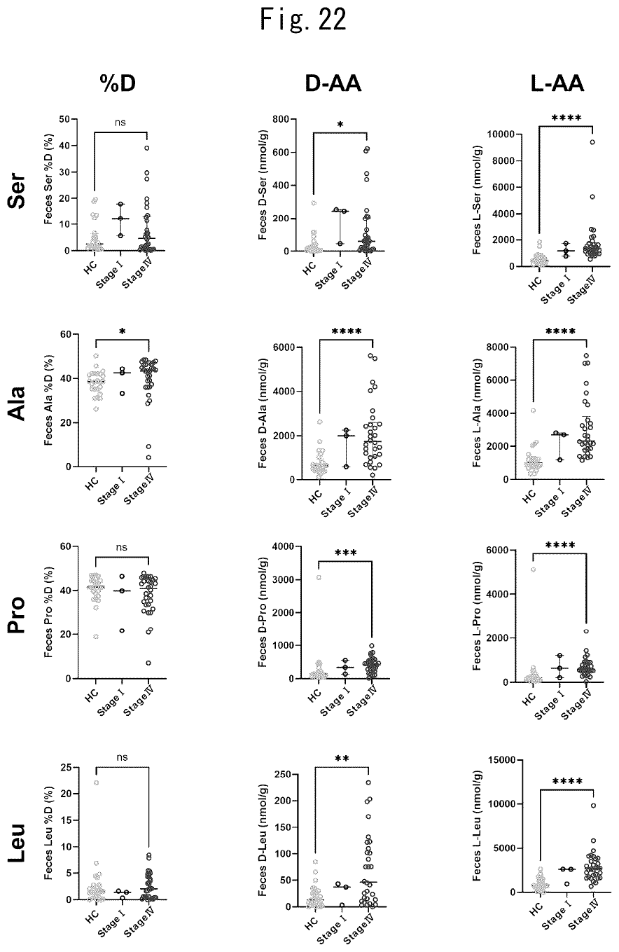

FD-AA and FL-AA were measured for 24 healthy subjects and 33 stomach cancer patients (Stage IV: 30 individuals, Stage I: 3 individuals), and F % D was calculated. FD-AA, FL-AA and F % D-Ala were found to increase with progressive cancer stage, providing information relating to stage classification (FIG. 22). Based on these results, highly precise assessment is possible by validation of cancer detection and diagnosis results, and stage classification, based on panel examination using an indicator based on multiple FD-AA, UD-AA and PD-AA.

### [Example 7] Demonstration and Validation of Example 1

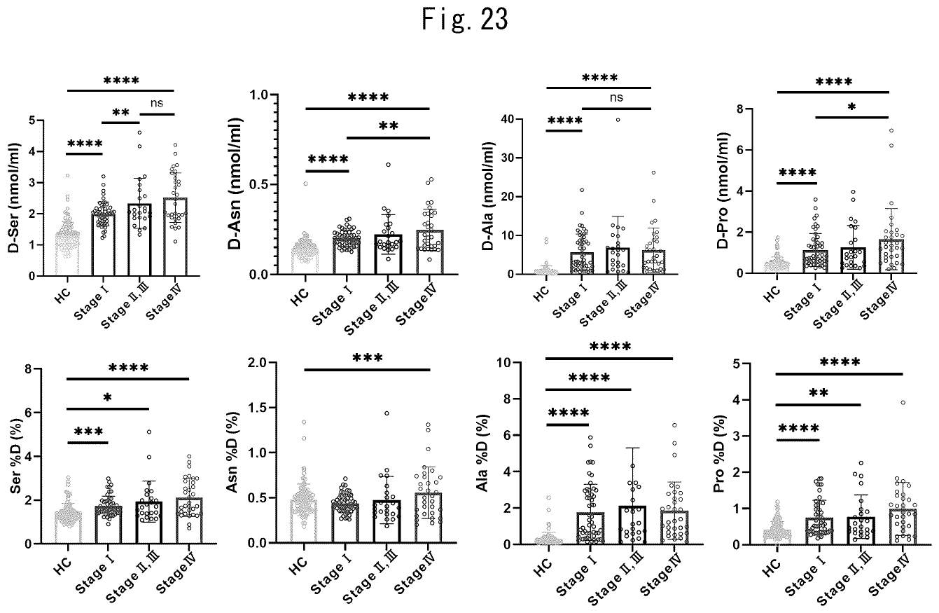

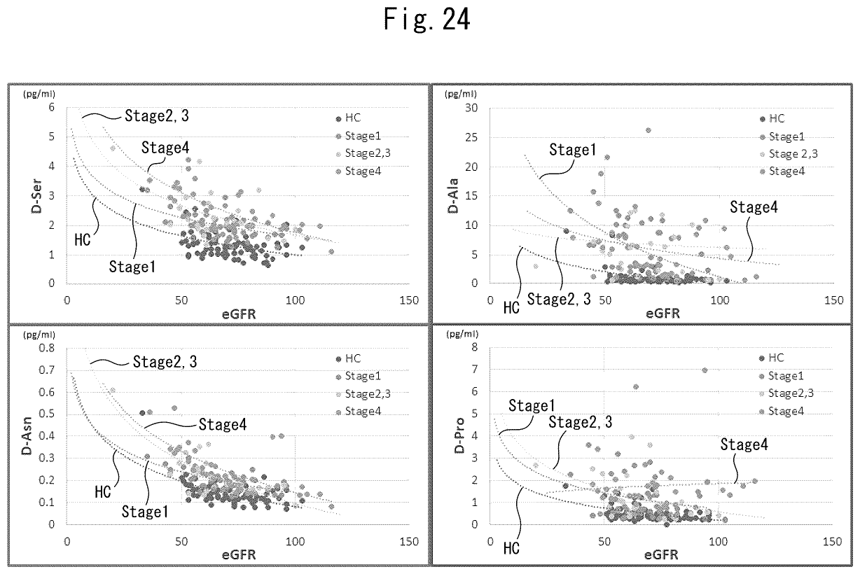

PD-AA for 84 healthy subjects and 137 stomach cancer patients (Stage I: 52 individuals, Stage II, III: 24 individuals, Stage IV: 32 individuals (all prior to treatment)) was validated for Examples 1 and 2 (FIG. 23). As a result, all PD-AA and P % D showed the same tendency as in Example 1 (FIGS. 10 and 11), thus demonstrating and validating the concept of assessment of cancer using an indicator based on PD-AA. This tendency was the same, even when corrected by kidney function (FIG. 24).

Each PD-AA, and especially PD-Ala and PD-Pro, showed significant increase from early Stage I, and based on the data for 84 healthy subjects and 52 patients with early gastric cancer, high-precision detection and classification, with AUC of 0.976, sensitivity of 92.3% and specificity of 98.8%, was possible for early gastric cancer in ROC curve analysis using kidney function-corrected values. Moreover, it was possible to provide precise information relating to cancer progression by limiting the data within a range of kidney function, and more specifically, PD-Ala and PD-Pro can classify progression at higher resolution for patients with relatively normal kidney function (eGFR>60). Prognosis was poor (P<0.001) for patients with high PD-AA (for example, D-Ser>3.0 nmol/mL) and relatively normal kidney function.

1. A method for providing information associated with cancer in a subject, using an indicator based on the amount of a D-amino acid in a biological sample of the subject, wherein the information is selected from the group consisting of:
a validation result for a cancer examination result or a cancer diagnosis result for the subject,
classification of cancer progression for the subject,
a result of cancer prognosis prediction for the subject, and
information for selection of cancer treatment means for the subject.

2. The method according to claim 1, wherein the indicator is a formula or value obtained by correcting the amount of the D-amino acid with an in vivo substance in the subject.

3. The method according to claim 2, wherein the in vivo substance is an L-amino acid.

4. The method according to claim 1, wherein the indicator is a formula or value obtained by correcting the amount of the D-amino acid with a kidney function marker of the subject.

5. The method according to claim 4, wherein the kidney function marker is the amount of one or more factors selected from the group consisting of creatinine, cystatin C, inulin clearance, creatinine clearance, urine protein, urine albumin, β2-MG, α1-MG, NAG, L-FABP and NGAL.

6. The method according to claim 1, wherein the D-amino acid is one or more selected from the group consisting of D-proline, D-serine, D-alanine, D-asparagine and D-leucine.

7. The method according to claim 1, wherein the cancer is gastrointestinal cancer.

8. The method according to claim 7, wherein the gastrointestinal cancer is stomach cancer, esophageal cancer or colorectal cancer.

9. The method according to claim 1, wherein the cancer treatment means is an antineoplastic agent.

10. The method according to claim 9, wherein the antineoplastic agent is an immune checkpoint inhibitor and/or an NMDA receptor antagonist.

11. The method according to claim 10, wherein the immune checkpoint inhibitor is an inhibitor of an immune checkpoint molecule selected from the group consisting of CTLA-4, PD-1, PD-L1, PD-L2, LAG-3, TIM3, BTLA, B7H3, B7H4, 2B4, CD160, A2aR, KIR, VISTA and TIGIT.

12. The method according to claim 1, wherein the validation result for the cancer examination result or the diagnosis result for the subject are provided by comparing the indicator with a determination value determined from the amounts of D-amino acids in biological samples from subjects having cancer.

13. The method according to claim 1, wherein the information for selection of cancer treatment means for the subject is provided by comparing the indicator with a determination value determined from the amounts of D-amino acids in biological samples from patients with cancer that respond and/or do not respond to cancer treatment means.

14. The method according to claim 1, wherein the information relating to classification of cancer progression for the subject is provided by comparing the indicator with a determination value determined from the amounts of D-amino acids in biological samples from patients with cancer whose cancer stage has been classified.

15. The method according to claim 1, wherein the information relating to prognosis prediction of cancer for the subject is provided by comparing the indicator with a determination value determined from the amounts of D-amino acids in biological samples from patients with cancer with information regarding prognosis.

16. A method for treating cancer, wherein a subject is treated by cancer treatment means selected based on information provided by the method according to claim 1.

17. The method according to claim 16, wherein the cancer treatment means consists of or comprises means for adjusting the amount of the D-amino acid in a biological sample of the subject, so that the value of an indicator based on the amount of the D-amino acid in the biological sample of the subject is within or near a prescribed range.

18. A system for providing information associated with cancer in a subject, comprising a memory unit, an input unit, an analytical measurement unit, a data processing unit and an output unit, wherein:
the memory unit stores the amount of a D-amino acid in a biological sample and a determination value associated with cancer, the determination value being selected from the group consisting of:
an determination value relating to the validity of cancer examination results or cancer diagnosis results determined from the amounts of D-amino acids in biological samples of patients with cancer; an determination value relating to cancer progression determined from the amounts of D-amino acids in the biological samples of the patients with cancer;
an determination value relating to cancer prognosis determined from the amounts of D-amino acids in the biological samples of the patients with cancer; and
an determination value for selection of cancer treatment means determined from the amounts of the D-amino acids in the biological samples of the patients with cancer;
the analytical measurement unit separates and quantifies the D-amino acid in the biological sample of the subject;
the data processing unit selects information associated with cancer in the subject by comparing an indicator based on the amount of the D-amino acid of the subject with the determination value stored in the memory unit; and
the output unit outputs the information.
